(19) United States
(12) Patent Application Publication
(10) Pub. No.: US 2023/0223118 A1
(43) Pub. Date: Jul. 13, 2023

(54) ADAPTIVE SPECIMEN COLLECTION ORDER PROCESSING

(71) Applicant: BioReference Health, LLC, Elmwood Park, NJ (US)

(72) Inventors: Richard SCHWABACHER, II, Elmwood Park, NJ (US); Vincent PACIONE, Campbell Hall, NY (US); Sandeep VIJAYAKUMAR, Burlington, CT (US); Ryan WIARANOWSKI, Loveland, OH (US)

(73) Assignee: BioReference Health, LLC, Elmwood Park, NJ (US)

(21) Appl. No.: 17/962,246

(22) Filed: Oct. 7, 2022

Related U.S. Application Data
(60) Provisional application No. 63/298,944, filed on Jan. 12, 2022.

(51) Int. Cl. G16H 10/40

(52) U.S. Cl. CPC G16H 10/40

(57) ABSTRACT

Disclosed herein are system, method, and computer program product embodiments for adaptive specimen collection order processing. An embodiment operates by retrieving a specimen order for processing a specimen collection. The specimen order comprises a test code. The embodiment determines that the specimen order is an eligible specimen order by determining that the test code matches at least one of a plurality of qualifying test codes. The embodiment transmits, to an electronic patient system, a request to provide resource allocation information for processing the specimen collection associated with the eligible specimen order. The embodiment generates an event for performing the specimen collection that satisfies the resource allocation information. The embodiment retrieves digital evidence that the event was completed. The embodiment validates that the event was completed based on the retrieved digital evidence. The embodiment then updates the eligible specimen order with a completion test code indicating the specimen collection was completed.

### CROSS-REFERENCE TO RELATED APPLICATION

This application claims the benefit of provisional U.S. Patent Application No. 63/298,944, titled “Adaptive Specimen Collection Order Processing” filed on Jan. 12, 2022, all of which is incorporated herein by reference in its entirety.

### BACKGROUND

Generally, laboratory diagnostics were not easily and readily available for specimen collection at home. With the introduction of the Coronavirus Disease 2019, there has been a steady demand for providing healthcare services directly at home. However, within the context of on-demand laboratory diagnostics, this need has generally not yet been met. On-demand diagnostics may be highly beneficial because it may reduce barriers for patients and healthcare providers, alike, to complete the providers' laboratory orders in an accessible and meaningful manner. However, on-demand diagnostics have generally not been readily available for patients, despite its demand. Additionally, on-demand diagnostics have generally not been introduced at a mass scale due to difficulties healthcare providers and laboratories face with integrating specimen collection order processing systems.

Specimen collection orders are often processed by interfacing with many different systems. Healthcare providers may process specimen collection results using and interfacing with one or more of electronic health record systems, specimen order management systems, digital specimen collection processing, and even outsource laboratory specimen collection to specimen collection processing systems designed particularly for specimen collection and drop off. Each of these systems generally have a particular integration. As a result, legacy specimen collection processing systems often must create a new integration compatible with each of the other systems with which it interfaces to provide the results of the specimen collection. The efforts expended to create new integrations compatible with each system are often duplicative, costly, and inefficient for specimen collection processing.

### BRIEF DESCRIPTION OF THE DRAWINGS

The accompanying drawings are incorporated herein and form a part of the specification.

FIG. 1 is a block diagram illustrating an adaptive specimen collection order processing system, according to some embodiments.

FIGS. 2A-2I are example graphical user interfaces illustrating interaction with an adaptive specimen collection order processing system, according to some embodiments.

FIG. 3 is a flowchart illustrating a method for adaptive specimen collection order processing, according to some embodiments.

FIG. 4 is an example computer system useful for implementing various embodiments.

FIG. 5 is a toolkit according to various embodiments.

FIG. 6 is the toolkit of FIG. 5.

FIG. 7 is the toolkit of FIG. 5.

FIG. 8 is a centrifuge processing station according to various embodiments.

FIG. 9 is the centrifuge processing station of FIG. 8.

FIG. 10 is the centrifuge processing station of FIG. 8.

FIG. 11 is the centrifuge processing station of FIG. 8.

FIG. 12 is a vehicle according to various embodiments.

FIG. 13 is a toolkit according to various embodiments.

FIG. 14 is a toolkit according to various embodiments.

FIG. 15 is a toolkit according to various embodiments.

FIG. 16 is a tray according to various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for adaptive specimen collection processing. With the introduction of Coronavirus Disease 2019, many services are now provided within the home. With many people staying at home and working remotely, the benefits of on-demand diagnostics are innumerable. On-demand diagnostics would be invaluable for patients who are unable to coordinate phlebotomies beyond the home. Generally, coordination between patients and healthcare providers for laboratory diagnostics were cumbersome.

For example, traditionally, on-demand diagnostics were reserved for certain patients that qualified for a “home draw.” A “home draw” was essentially coined as the term for collecting and dropping off specimens within the patient's home. While laboratories would offer this as a service, this was not readily available or advertised. Patients were generally required to coordinate a “home draw” with a healthcare provider in a complex manner, for example, using designated personnel to coordinate the “home draw.” Therefore, on-demand diagnostics was not readily available to the masses, despite its crucial need given the rising demand for on-demand healthcare services in light of the Coronavirus Disease 2019 pandemic.

Additionally, legacy specimen collection processing providers pose barriers for healthcare providers to seamlessly adopt and integrate specimen collection processing into their existing electronic health record systems, specimen order management systems, and other healthcare electronic systems. There are many variations of system interfacing in order to process specimen collection orders. Each system may require different formats for specimen collection orders.

As a first example, for illustrative purposes, a small healthcare provider, such as a doctor's office, may use an electronic health record system. An electronic health record system may be a system that stores real-time patient records, such as, a patient's medical history, diagnoses, medications, treatment plans, laboratory test results, etc. In some circumstances, healthcare providers in a doctor's office do not complete specimen collection orders themselves. In these circumstances, small healthcare providers or traditional doctor's offices may not have in-house services to process specimen collection orders, so small healthcare providers may outsource specimen collection orders to laboratories. As a result, organizations managing specimen collection order processing often must create their own integration compatible with the small healthcare provider's electronic health record system. Moreover, each small healthcare provider may have a different electronic health record system. Therefore, a different and direct integration may need to be created to be compatible with each and every small healthcare provider's electronic health record system. This can lead to duplicative and costly efforts for organizations processing specimen collection orders.

As another example, for illustrative purposes, a large healthcare system containing multiple hospitals may need to outsource specimen collection orders. In some cases, an organization processing specimen collection orders may be involved as a reference laboratory. For example, many hospitals may have a pre-compiled list of specimen collection orders the hospital's in-house laboratory can process. However, sometimes a hospital's in-house laboratory may be unable to process certain specimen collection orders. In these cases, a hospital may need to outsource specimen collection orders that its in-house laboratory is unable to process. These hospitals may refer this work to a reference laboratory that processes specimen collection orders.

In this case, a healthcare provider operating within an electronic health record system may transmit a request to the specimen order management system and/or a laboratory information system of the hospital. A specimen order management system and/or laboratory information system may be a system that processes, stores, and manages patient data related to laboratory processes and specimen collection. These systems may manage this information to coordinate the workflow of in-house and outsourced specimen collection order processing.

Once the specimen order management system and/or the laboratory information system retrieves a specimen collection order, the hospital may make a decision on what the specimen collection orders that can be processed in-house and the specimen collection orders that will need to be outsourced to the reference laboratory. As a result, the reference laboratory may need to create a direct integration to the specimen order management system and/or laboratory information system in order to receive specimen collection orders and send back specimen collection results. Again, each large healthcare provider may have a different specimen order management system and/or laboratory information system. Therefore, a different and direct integration may need to be created to be compatible with each and every large healthcare provider's specimen order management system and/or laboratory information system. This can lead to duplicative and costly efforts for organizations processing specimen collection orders.

Regardless of the size and nature of the healthcare provider, organizations managing and processing specimen collection orders may need to create a particular integration for each system with which it is interfacing. This may involve formatting the specimen collection order in a particular format for each system with which it is interfacing. This can be extremely cumbersome, costly, duplicative, and confusing for both organizations processing specimen collection orders and those receiving the results of specimen collection orders.

Embodiments described herein solve the above-mentioned technological problems by providing adaptive specimen order collection. An embodiment operates by retrieving a specimen order for processing a specimen collection. The specimen order comprises a test code. The embodiment determines that the specimen order is an eligible specimen order by determining that the test code matches at least one of a plurality of qualifying test codes. The embodiment transmits, to a patient system, a request to provide information for processing the specimen collection associated with the eligible specimen order. The embodiment generates an event for performing the specimen collection. The embodiment retrieves digital evidence that the event was completed. The embodiment validates that the event was completed based on the retrieved digital evidence. The embodiment then updates the eligible specimen order with a completion test code indicating the specimen collection was completed.

In view of the above, embodiments described herein reduce the barrier to adoption of specimen collection order processing with the use of test codes. For example, a healthcare provider can provide a test code with the specimen collection order indicating which operational workflow they desire to process the order. Upon receipt of the test code, a specimen collection order processing system can route a specimen order to different systems to process the specimen collection order without organizations having to create a separate integration compatible with each system.

Furthermore, the healthcare provider may issue the specimen collection order in a non-standardized format (e.g., an image of the specimen collection order). That is, the healthcare provider may not need to format the specimen collection order into a particular format for processing. The specimen collection order may be routed to the appropriate specimen processing module using the test code.

Each time a new integration is implemented in a healthcare provider's system, a healthcare professional may need to log into separate systems and learn how to manage and operate in the new system for reviewing specimen collection orders. However, with the introduction of test codes designated with each specimen collection order, healthcare providers would not need to operate an entirely new integration in their systems just for reviewing specimen collection orders. Therefore, the use of test codes with specimen collection orders provides a technological solution to a technical problem healthcare providers' face by eliminating the need to integrate a new system for specimen collection order processing. Moreover, the use of test codes may eliminate the need to create a tailored integration for each system. As such, the use of test codes may provide an improvement to the functioning of a computer by conserving computational and operational resources.

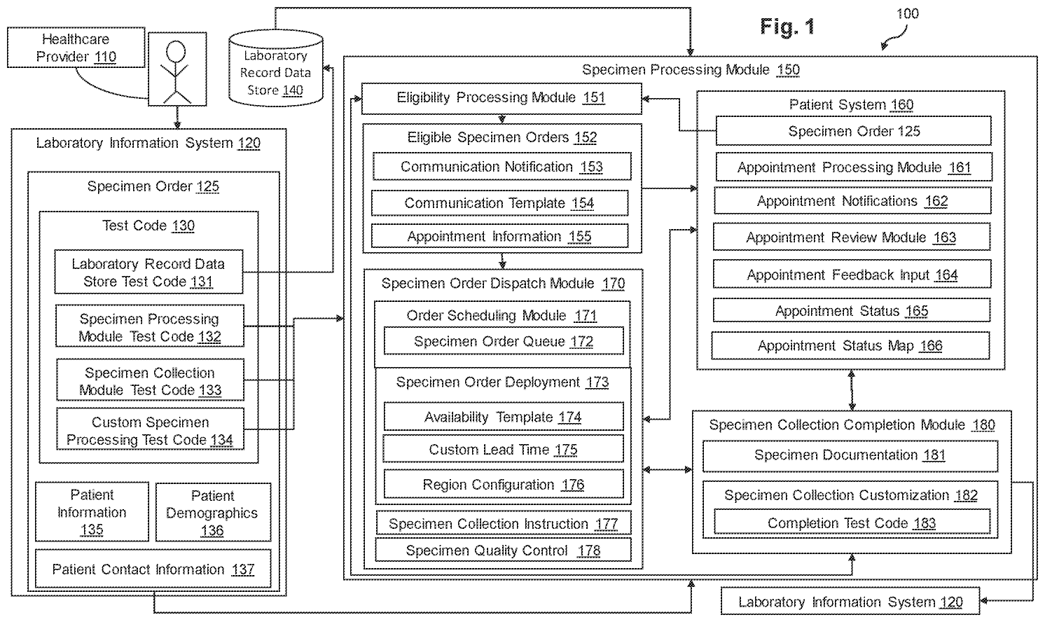

FIG. 1 is a block diagram illustrating an adaptive specimen collection ordering system, according to some embodiments. The system 100 is an example embodiment of an adaptive specimen collection ordering system. System 100 may include a laboratory information system 120, laboratory record data store 140, and specimen processing module 150. These components may be connected together through one or more networks. A healthcare provider 110 may access system 100 to place an adaptive specimen collection order based on a patient's needs. Healthcare provider 110 may place an adaptive specimen collection order by providing relevant information as an input to laboratory information system 120. Specimen collection may include any manner of collecting a specimen, including, but not limited to, phlebotomies, urinalysis, salivaomics, etc. Laboratory information system 120 may be a graphical user interface through which the healthcare provider 110 submits a specimen order 125 as an input. Specimen order 125 may be a specimen collection order with information including, but not limited to, test code 130, patient information 135, patient demographics 136, and patient contact information 137.

Test code 130 may be a unique test code for specimen processing module 150 to adaptively route a specimen collection order based on the specific processing needs of the specimen order 125 itself. For example, healthcare provider 110 may input a laboratory record data store test code 131 as part of a specimen order 125. This can avoid the healthcare provider 110 having to operate an entirely new integration for reviewing specimen collection results.

Test code 130 may be laboratory record data store test code 131, automated specimen processing module test code 132, specimen collection module test code 133, or custom specimen processing test code 134. Test code 130 may be other types of test codes as would be appreciated by a person of ordinary skill in the art. Laboratory record data store test code 131 may be a test code indicating that specimen order 125 is to be handled by specimen processing module 150. Laboratory record data store test code 131 may be used when a healthcare provider 110 wants to selectively choose when and for which specimen orders 125 will be transmitted to specimen processing module 150. In some embodiments, laboratory information system 120 may access laboratory record data store 140 to identify laboratory record data store test code 131. For example, laboratory record data store 140 may be a database that stores one or more laboratory record data store test codes 131. Healthcare provider 110 may select laboratory record data store test code 131 from a list of available laboratory record data store test codes 131 stored in laboratory record data store 140. Laboratory information system 120 may include a graphical user interface allowing healthcare provider 110 to perform this selection. The selection may occur when healthcare provider provides specimen order 125 and/or when identifying test code 130.

Accordingly, if a specimen order 125 contains laboratory record data store test code 131, laboratory information system 120 may transmit that particular specimen order 125 to specimen processing module 150 for handling. Therefore, because laboratory record data store test code 131 is provided within specimen order 125, healthcare provider 110 may need to provide a laboratory record data store test code 131 within another specimen order 125 if that other specimen order 125 is to be handled by specimen processing module 150. In some embodiments, specimen processing module 150 may retrieve data corresponding to the specimen order 125 from laboratory record data store 140 in response to receiving laboratory record data store test code 131. This data may include adaptively routing data for specimen collection corresponding to laboratory record data store test code 131.

Specimen order 125 may also contain an automated specimen processing module test code 132. In many circumstances, healthcare providers 110 may transmit specimen order requests 125 to different systems. However, if a healthcare provider 110 anticipates that all specimen order 125 will ultimately be processed by specimen processing module 150, healthcare provider 110 may provide automated specimen processing module test code 132. As a result, healthcare provider 110 may not need to provide a laboratory record data store test code 131. Instead, healthcare provider 110 may provide only automated specimen processing module test code 132. Accordingly, when laboratory information system 120 receives a specimen order 125, laboratory information system 120 may determine that the specimen order 125 is associated with a customer or account number of the healthcare provider 110. Laboratory information system 120 may determine that the healthcare provider 110 provided automated specimen processing module test code 132. As such, laboratory information system 120 may automatically route the specimen order 125 to specimen processing module 150. As a result, the healthcare provider 110 may not need to provide a test code 130 for every specimen order 125. This configuration can allow for laboratory information system 120 to seamlessly route specimen orders 125 to specimen processing module 150 without requiring any additional information from the healthcare provider 110. By doing so, laboratory information system 120 can require fewer computational resources for routing specimen order 125.

Specimen order 125 may also contain a specimen collection module test code 133. Specimen collection module test code 133 may be used to enable a workflow for specimen collection and drop-off. If healthcare provider 110 anticipates that specimen orders will ultimately be processed through specimen processing module 150, but is limited to specimen collection and drop-off, healthcare provider 110 may provide specimen collection module test code 133 to indicate specimen processing module will only process specimen order 125 for specimen collection and drop-off.

Specimen collection module test code 133 may include a “Date of Collection” parameter as part of specimen order 125 to streamline specimen collection and drop-off. Therefore, if specimen order 125 contains specimen collection module test code 133, laboratory information system 120 may transmit specimen order 125 directly to specimen processing module 150 for the purpose of processing specimen collection and drop-off.

Therefore, without expending additional resources for processing specimen order 125, laboratory information system 120 may transmit specimen order 125 directly to specimen processing module 150 for a narrow processing purpose (e.g., specimen collection and drop-off). That is, use of specimen collection module test code 133 may allow laboratory information system 120 to prevent any additional action to be executed on specimen order 125 other than specimen collection and drop-off. As a result, specimen collection and drop-off processing may require fewer computational resources. Moreover, use of specimen collection module test code 133 can prevent use of extraneous computational resources, thereby improving the functioning of the computer itself.

In some embodiments, test code 130 may be a custom specimen processing test code 134. For example, a healthcare provider 110 may define a custom specimen processing test code 134 for specimen processing module 150 to use to adaptively route a specimen collection order. Healthcare provider 110 may pre-define and/or customize custom specimen processing test code 134 for subsequent use.

Specimen processing module 150 may be a master data repository for all laboratory specimen orders 125. Specimen processing module 150 may be a relational database, a NoSQL database or other horizontally scaling database, or any other database adhering to a suitable database design methodology. In some embodiments, specimen processing module 150 may implement a centralized storage area network (SAN), network-attached storage (NAS), redundant array of independent disks, and/or any other configuration of storage devices to supply sufficient storage capacity to store database tables and supporting structures. Sufficient storage may alternatively exist in any other physically attached magnetic storage, cloud storage, or additional storage medium. In some embodiments, specimen processing module 150 may deploy a hard-disk interface, such as ATA, SATA, SCSI, SAS, and/or fibre for interfacing with storage mediums.

As described above, integrating multiple systems with each laboratory information system 120 may be cumbersome, inefficient, and require a large amount of computational resources. By retrieving test code 130, system 100 may adaptively route a specimen collection order based on the specific needs of a healthcare provider 110 without needing to create a separate system compatible with each laboratory information system 120.

Once specimen processing module 150 retrieves a specimen order 125, eligibility processing module 151 determines whether specimen order 125 can be processed based on its test code 130. In some embodiments, eligibility processing module 151 may consider patient demographics and/or a requested service location to determine eligibility. For example, specimen processing module 150 may identify whether test code 130 matches a qualifying test code and/or satisfies patient and/or location rules or parameters corresponding to test code 130. Eligibility processing module 151 can compare the test code 130 to test codes eligible for processing through specimen processing module 150, including, but not limited to, laboratory record data store test code 131, automated specimen processing module test code 132, or specimen collection module test code 133.

If eligibility processing module 151 determines the test code 130 matches one of the qualifying test codes and/or satisfies the patient and/or location rules or parameters previously discussed, eligibility processing module 151 may denote specimen order 125 as an eligible specimen order 152. An eligible specimen order 152 may represent a specimen order 125 that is capable of being processed. Eligibility processing module 151 can then transmit a communication notification 153 based on the eligible specimen order 152 to a patient (e.g., to patient system 160).

Specimen processing module 150 may determine what to include in the communication notification 153 being transmitted to the patient (e.g., to patient system 160) using a communication template 154. For example, specimen processing module 150 may include appointment information 155 in the communication notification 153 based on the communication template 154. The appointment information 155 may include one or more appointment links for the patient to schedule an appointment for the specimen collection related to the eligible specimen order 152. Specimen processing module 150 may include other information associated with specimen processing module 150 in the communication notification 153 based on the communication template 154. Specimen processing module 150 may use communication template 154 to tailor the information in the communication notification 153 to a particular patient.

Specimen processing module 150 may also determine how to format the communication notification 153 being transmitted to the patient (e.g., to patient system 160) using the communication template 154. Specimen processing module 150 may use communication template 154 to tailor the format of information in the communication notification 153 to a particular patient.

Specimen processing module 150 may transmit the communication notification 153 to the patient (e.g., to patient system 160) via email, SMS, or another communication mechanism as would be appreciated by a person of ordinary skill in the art.

Interfacing or interconnection among various systems and layers, e.g., laboratory information system 120, and specimen processing module 150 may employ any number of mechanisms, such as any number of protocols, programmatic frameworks, or application programming interfaces (API). In some embodiments, laboratory information system 120 and specimen processing module 150 may connect with APIs. APIs may be configured with a particular service type, including, but not limited to, JSON (JavaScript Object Notation), Representational State Transfer (REST or RESTful web services), Extensible User Interface Protocol (XUP), Simple Object Access Protocol (SOAP), XML Schema Definition (XSD), XML Remote Procedure Call (XML-RPC), Message Queue (MQ), or any other service protocols known to a person of ordinary skill in the art.

Under the Health Insurance Portability and Accountability Act of 1996 (HIPAA) law, it is crucial to protect a patient's data by implementing rigorous cybersecurity measures. Accordingly, a healthcare provider 110 may provide information for sending an API request from laboratory information system 120 to specimen processing module 150 for particular information related to an eligible specimen order 152. Specimen processing module 150 may be configured with particular functional calls to securely provide patient information related to the eligible specimen order 152 to protect patient's privacy and confidentiality. The functional calls in specimen processing module 150 may easily be modified. This can reduce and narrow the amount of functional calls a laboratory information system 120 may access. As a result, not all functional calls are exposed to laboratory information system 120, which may significantly reduce the risk of a cybersecurity and/or privacy attack for any one of these systems.

Patient system 160 may be a graphical user interface through which a patient schedules and/or coordinates a specimen order 125. Patient system 160 may implement a web application. In some embodiments, patient system 160 may provide a graphical user interface corresponding to an Internet browser accessing a web application. Patient system 160 may be implemented on a computing device such as a laptop, PC, tablet, smartphone, and/or other device providing Internet access. Patient system 160 may include an appointment processing module 161, appointment notifications 162, appointment review module 163, appointment feedback input 164, appointment status 165, and appointment status map 166. In order to schedule an appointment for a specimen order 125, a patient may access an appointment link via email and/or SMS in the appointment processing module 161. A patient may choose a date and time of an appointment and choose a location, such as, for example, the patient's home or office workplace, for the specimen order 125 within the appointment processing module 161. Based on the patient demographics 136, the appointments in the appointment processing module 161 may be presented to a patient as appointment slots based on health professionals assigned to the patient's geographic demographics and the availability of the health professionals to perform specimen collection for the selected date and time.

While scheduling the specimen collection, a patient may opt-in to receive appointment notifications 162 that may include notifications related to an eligible specimen order 152. Specimen processing module 150 may configure appointment notifications 162 and may retrieve patient's consent to receive appointment notifications for compliance with legal requirements. A patient, through the appointment review module 163, may review the details of the upcoming appointment for the specimen collection, opt-in to receive appointment notifications 162, and provide appointment feedback input 164, such as information related to appointment location and other relevant information a patient may want to share in advance of the scheduled appointment, to enable a health professional to better prepare for the appointment. A patient may then submit the appointment for the associated eligible specimen order 152.

Upon the patient submitting the appointment, the specimen order dispatch module 170 may retrieve the appointment for the eligible specimen order 152. Specimen order dispatch module 170 may be a backend of system 100 configured to process dispatching the eligible specimen order 152. Specimen order dispatch module 170 may include an order scheduling module 171, specimen order deployment 173, and/or a specimen collection instruction 177. Order schedule module 171 may process eligible specimen orders 152 using a specimen order queue 172. Health professionals may configure incoming eligible specimen orders 152 by configuring the position of these orders in a specimen order queue 172. Healthcare providers may configure specimen order queues 172 to initiate the operational workflow for the scheduled specimen collection. Health professionals may provide their location, availability, and other information to enhance specimen collection dispatch.

Once the appointment is retrieved, specimen order dispatch module 170 may create an object representing a job, according to some embodiments. System 100 may determine which health professionals is suitable for fulfilling the specimen collection appointment. Order schedule module 171 may consider the availability and other information of healthcare professionals to allocate the job corresponding to the retrieved eligible specimen order 152. Order scheduling module 171 may configure the specimen order queue 172 to eliminate any overlapping specimen collection appointments among health professionals.

In addition to information related to health professionals, specimen order dispatch module 170 may also use information retrieved from or related to a patient to configure specimen order deployment 173. Specimen order deployment 173 may consider the location of the patient to configure specimen order queue 172 using region configuration 176. Region level zip codes are loaded onto the backend of specimen processing module 150 within specimen order dispatch module 170. Specimen order deployment 173 may use availability templates 174 configured to determine whether specimen processing module 150 is serviceable in a particular region based on whether any health professional is available in the region.

Using the address and zip code retrieved from the patient, region configuration 176 may determine whether a health professional is aligned to the corresponding region. In some embodiments, the patient's device's global positioning system (GPS) location may be used by region configuration 176 to determine whether a health professional is in proximity (e.g., within a threshold traveling distance or amount of travel time) to the corresponding region.

Based on this determination, specimen order deployment 173 may provide feedback regarding whether specimen processing module 150 is serviceable in the provided region for the scheduled specimen collection. Region configuration 176 may also customize region configurations to provide flexible scheduling based on the needs of a specific region. Additionally, specimen order deployment 173 may include custom lead time 175 in which regions are set with a custom lead-time that provides additional travel time as a buffer for health professional traveling to the patient for a scheduled specimen collection, which allows for smooth resource scheduling.

Additionally, specimen order dispatch module 170 may include a specimen collection instruction 177 to configure the workflow of specimen collection and drop-off. Accordingly, for a health professional to perform the specimen collection, specimen collection instruction 177 may retrieve guidance and instructions for the health professional performing specimen collection and or drop-off.

Specimen order dispatch module 170 may also include a module for specimen quality control 178. Specimen order dispatch module 170 may schedule the appointment for an eligible specimen order 152 based on whether the specimen would retain its quality. Specimen order dispatch module 170 may analyze the potential quality of the specimen based on the region in which the patient is located. For example, considering the temperature in the region, specimen order dispatch module 170 may determine a timeframe within which the specimen would need to be collected and dropped off. Based on this timeframe, specimen order dispatch module 170 may determine a health professional that would be able to perform the scheduled specimen collection within that timeframe while the specimen is still able to retain its quality.

If specimen order dispatch module 170 determines that a health professional was previously scheduled for the specimen collection, but that the specimen would lose its quality based on the health professional's location and other information, specimen order dispatch module 170 may determine another health professional that would be able to perform the specimen collection while the specimen retains its quality. Specimen order dispatch module 170 may consider any number of factors known to a person of ordinary skill in the art to determine how to schedule a health professional for the ordered specimen collection in order to retain and control the quality of the specimen.

After processing the appointment for an eligible specimen order 152, specimen order dispatch module 170 may transmit information related to the scheduled appointment for the eligible specimen order 152 to patient system 160. Patient system 160 may include an appointment status 165 and/or appointment status map 166 to apprise the patient of information related to the upcoming specimen collection. Appointment status 165 may be configured to notify the patient with confirmation details of the appointment for the eligible specimen order 152 and a link to a page with the appointment status 165. The link may display instructions for the patient related to the upcoming specimen collection appointment and may also include contact information for a health professional. Appointment status 165 may display the timeline and progress of fulfilling the specimen collection appointment for the eligible specimen order 152.

Prior to the scheduled specimen collection appointment, patient system 160 may update appointment status 165 of the eligible specimen order 152 as the health professional proceeds to the patient's location. The patient may receive notifications as the health professional is en-route to the patient's location to perform the scheduled specimen collection. The patient may access an appointment status map 166 that displays the location of the health professional in a map. Patient system 160 may update and display the updated location of the health professional and the patient may track the health professional in real-time within appointment status map 166. Patient system 160 may transmit a notification as the health professional arrives at the patient's location. Patient system 160 may also transmit various notifications based on the health professional executing different events for completing the eligible specimen order 152.

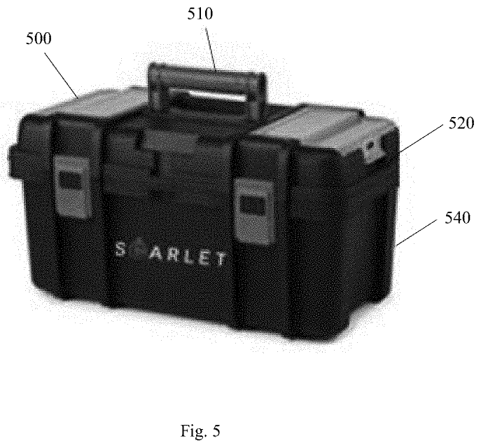

Once the health professional has arrived at the patient's location, the health professional may perform the scheduled specimen collection with the use of a toolkit 500 and a storage 600 (FIG. 8). As shown in FIGS. 5-7, toolkit 500 may contain one or more supplies 550 for collecting one or more specimens. Toolkit 500 may include a handle 510, a top portion 520, and a bottom portion 540. Transitioning specimen collection from sedentary environments to mobile environments may pose many challenges with respect to the patient experience and obtaining quality results. Along with toolkit 500, a health professional may be provided with educational materials regarding toolkit 500, storage 600 (FIG. 8), and/or methods for specimen collection. These materials, or standard operating procedures (SOPs), may facilitate seamless specimen collection to remedy the challenges of mobile specimen collection. SOPs are compliant with the standards and guidelines developed by the Clinical & Laboratory Standards Institute and instep with regulatory requirements that govern our operations.

Toolkit 500 may be functional in non-medical environments, such as homes and workplaces. Any supplies 550 required for the scheduled specimen collection may be contained. Additionally, a health professional may be instructed on how to use toolkit 500 safely and efficiently via one or more SOPs. SOPs may define the requirements for toolkit 500 to ensure compliance with proper specimen collection. Toolkit 500 may be self-contained, for example, by securely containing all supplies 550 needed for the scheduled specimen collection. In addition, toolkit 500 may be constructed to prevent crossover contamination of collected specimens. Each of supplies 550 and any collected specimens may be arranged within toolkit 500 in an organized and secure manner to maintain the integrity of the same. The exterior and interior surfaces of toolkit 500 may comprise a plastic or a coating material to keep toolkit 500 and its components clean and dry. The material of toolkit 500 may also permit easy cleaning and wipe down after specimen collection. Toolkit 500 may also be comprised of an impermeable material to prevent contamination. Because specimen collection appointments are in non-medical environments, it may be helpful to have a discrete toolkit 500 and supplies 550. Health professionals may easily carry toolkit 500 if it is smaller, and some non-medical environments may better support smaller toolkits 500. For example, environments that are compact and/or crowded may benefit from smaller toolkits 500. Thus, the arrangement and organization provided by toolkit 500 may reduce the size needed for toolkit 500.

Providing toolkit 500 in this way may be advantageous for non-medical environments that may be susceptible to risk with respect to specimen collection. This may introduce contaminants into the environment, such as by contacting surfaces and/or others. Retrieving and storing a specimen without exposing it to potentially contaminated environments may facilitate effective and safe specimen collection. Patients may experience seamless specimen collection as a result.

Other aspects that may influence the patient experience may include collection time. Toolkit 500 may be optimized to provide health professionals with quick and easy access to applicable supplies 550. In this way, patients may be able to provide a specimen and return to their day with minimal disruption. In contrast, a dedicated lab service location may require patients to travel and wait for specimen collection. Health professionals using toolkit 500 may be provided with SOPs to consider these variables in various non-medical environments. For example, homes or workplaces may be crowded or noisy. Additionally, children and/or pets may cause activity in the vicinity of a specimen collection appointment, which may be distracting. Accordingly, SOPs may require health professionals to be organized and efficient to safely and effectively collect specimen. SOPs may provide instructions on safe collection and how to minimize distraction.

Another aspect that may influence the patient experience is safety. Safe collection may be facilitated by providing instructions to that end via SOPs. For example, a health professional may be instructed on retrieval of supplies 550 from toolkit 500, usage of supplies 550 with patients, and return of supplies 550 to toolkit 500. Supplies 550 may be accounted for throughout a specimen collection appointment. Accordingly, supplies 550 that may be unsafe to handle (e.g., needles) are controlled by the health professional to prevent injury.

Setting up toolkit 500 may include receiving, organizing, and packing supplies 550. A health professional may receive supplies 550 in one or more shipments. The inventory of supplies 550 may include supplies 550 specific to an eligible test. Examples of eligible tests categories include venipuncture, urine, and Covid-19 polymerase chain reaction (RT-PCR) collections. Frequently ordered tests include comprehensive metabolic panel (CMP), complete blood count (CBC), hemoglobin A1c, thyroid stimulating hormone (TSH), and Covid-19 RT-PCR. Eligible tests include tests for which collection of appropriate specimen is facilitated by the systems and methods described herein. These tests require following standards and regulations for laboratory testing and may incorporate endocrine/thyroid functions, general health screening, specific diagnostic monitoring, allergens, etc. Test-specific supplies are provided along with SOPs on storage requirements to maintain specimen integrity, which may include information on temperature, for example. Many supplies 550 may be applicable to multiple tests and may be cross-utilized as many tests have the same or similar collection requirements.

One or more of the shipments may contain an inventory list that may be cross-referenced with the received supplies 550 to ensure that the health professional received all the supplies 550 listed in the denoted quantities. The shipping containers themselves may be stored for subsequent use, such as if one or more supplies 550 need to be returned to the supplier.

After conducting an inventory check of the received shipment(s), a health professional may organize supplies 550 within toolkit 500. Not all supplies 550 may be required for a scheduled specimen collection. Therefore, a storage location may be designated to store excess inventory. The health professional may identify and designate a clean, dry, and secure location to safely store any excess inventory. SOPs may provide information on temperature requirements. Additionally, a health professional may be instructed to maintain supplies 550 in a neat area free of clutter. Like supplies 550 may be maintained together to keep the designate storage area organized. For example, personal protective equipment (PPE) and test tubes may each be grouped and stored together exterior to toolkit 500 for organization, accessibility, and later inclusion in toolkit 500. The inventory list(s) provided in the received shipment(s) may also be maintained at the designated storage location to assist the health professional in periodically assessing inventory stock levels to resupply toolkit 500.

Prior to packing supplies 550 and/or prior to the scheduled specimen collection, the surfaces of toolkit 500 may be decontaminated. A health professional may periodically clean and decontaminate toolkit 500, to ensure that toolkit 500 may be safely brought into non-medical environments during specimen collection. SOPs may instruct a health professional on how to clean surfaces of toolkit 500. Additionally, prior to a scheduled specimen collection (e.g., the night before), the health professional may review the upcoming specimen collection and information related to any supplies 550 required for conducting the collection. After reviewing the requisitions of every upcoming specimen collection, the health professional may pack toolkit 500 with adequate supplies 550 for the tests ordered for each collection. Post-collection of the specimen, the health professional may replace any used supplies 550 from the health professional's designated storage location and/or obtain a new shipment of inventory.

Packing toolkit 500 may require a health professional to follow one or more SOPs. SOPs may instruct a health professional to first decontaminate toolkit 500 and/or review the upcoming specimen collection and information related to any supplies 550 required for conducting the specific specimen collection. Additionally, SOPs may direct the health professional to add and organize supplies 550 in specific areas within toolkit 500. Supplies 550 may be organized by utilization according to some embodiments. Utilization may include increasing efficiency and minimizing error and delays. For example, supplies 550 may be organized so that everything needed for the collection is easily retrievable by the health professional during the specimen collection. This may include having supplies 550 be visible and/or arranged within arm's length for the health professional. Based on the scheduled specimen collection, this organization may be modified. If a particular specimen collection requires select supplies 550, these priority supplies 550 may be readily available within toolkit 500, rather than buried under other supplies 550 or hidden from view. Organizing by utilization may lessen the burden on health professionals to provide quick and seamless collection. Patients may also experience greater satisfaction, as collection may be simple and personalized.

Organizing supplies 550 may also take into consideration weight distribution of supplies 550 within toolkit 500, overall weight of toolkit 500, and toolkit 500 dimensions for holding supplies 550 within toolkit 500. Further, organizing supplies 550 within toolkit 500 may take into consideration needs of the health professional. For example, if the health professional is left-handed, priority supplies 550 may be organized to be easily reachable by the health professional's left hand. The patient's needs may be similarly considered. For example, if the patient is left-handed and the scheduled collection requires drawing blood, the patient may desire for blood to be drawn from their right, or non-dominant, arm. Toolkit 500 may be organized to allow the health professional to best collect while being positioned on the right side of the patient. SOPs may additionally instruct the health professional on how and where to sit or stand to conduct the specimen collection. For example, the patient being seated on a stool may introduce risk into the specimen collection process or specimen collection. In contrast, the patient sitting on a chair with a back and arm rests may facilitate proper specimen collection.

A health professional may also be instructed on securing toolkit 500 before visiting a non-medical environment and after specimen collection. Contaminating the collected specimen, as well as securing the chain of custody of collected specimen are concerns when conducting a mobile operation. Toolkit 500 may be constructed to facilitate security and authorization. For example, toolkit 500 may include locking mechanisms that are operable by authorized users only, such as the healthcare professional that services toolkit 500. SOPs may provide instructions to actuate a locking mechanism of toolkit 500 during travel and when not in use for specimen collection. Accordingly, the health professional may lock toolkit 500 after packing it with supplies 550 and after collecting specimen. More than one health professional may be authorized to access the same toolkit 500. For example, multiple health professionals dispatched to the same location may be authorized to access toolkit 500. Multiple health professionals may be dispatched if additional support is required.

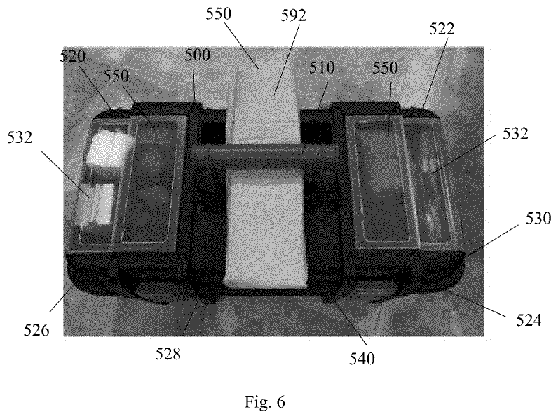

With reference to FIG. 6, toolkit 500 top portion 520 may include a top end 522, a bottom end 524, a first side region 526, a central region 528, and a second side region 530. First side region 526 and second side region 530 may be recessed in top portion 520. Handle 510 may extend through top portion 520 at central region 528.

Several supplies 550 may be included in toolkit 500. These supplies 550 may include cross-utilized supplies 550 and supplies 550 specific to the scheduled specimen collection. For example, supplies 550 added to toolkit 500 may include one or more of a container 552 (e.g., for sharps), a tube rack 554, a band 568 (e.g., a tourniquet), first needles 570 (e.g., black needles), second needles 572 (e.g., green needles), an infusion set 574 (e.g., a butterfly), gloves 576, an alcohol swab 578, gauze 580, a needle holder 582, a mask 584 (e.g., a disposable mask), adhesive 586 (e.g., a paper tape roll), hand sanitizer 588, a specimen container 590 (e.g., a small bag), a chux pad 592, a biohazard container 594 (e.g., a drawstring biohazard bag or green bag), and/or PPE 596. These supplies 550 may be arranged in one or more containers.

Supplies may be applicable to multiple tests and may be cross-utilized for specimen collections. Needles, gauze, alcohol swab, needle holder, tape, tubes, and tourniquets may be used for venous collections, for example. Some tests may require additional processing beyond specimen collection, such as centrifuging and aliquoting plasma. Accordingly, additional supplies may be required for different tests. Needle selection may be based on venous sufficiency. Needles, for example, first needles 570 and second needles 572, may have different sizes, or gauges. First needles 670 may be small gauge needles (e.g., 22 gauge (G) black needles) to facilitate draws in pediatric or small veins. Second needles 572 may be standard needles (e.g., 21G standard needles). Infusion set 574 may be used for more challenging collections.

Generally, supplies 550 for a scheduled specimen collection may be contained within toolkit 500 in bottom portion 540. When not being prepared for a scheduled specimen collection, toolkit 500 may store supplies 550 in top trays 532, for example excess supplies 550. One or more top trays 532 may be disposed on top end 522 and may be removable from toolkit 500 according to some embodiments. Top trays 532 may include bottom ends 534 that are received in the recessed first side region 526 and second side region 530. Prior to the scheduled specimen collection, top trays 532 may be removed and kept at the designated storage location for maintaining excess inventory.

To pack toolkit 500, bottom portion 540 is uncovered by removing top portion 520. Bottom portion 540 may include a first side region 542, a central region 544, and a second side region 546. The composition of bottom portion 540 is described below. It should be understood that the order of presentation is not necessarily the order in which supplies 550 may be placed.

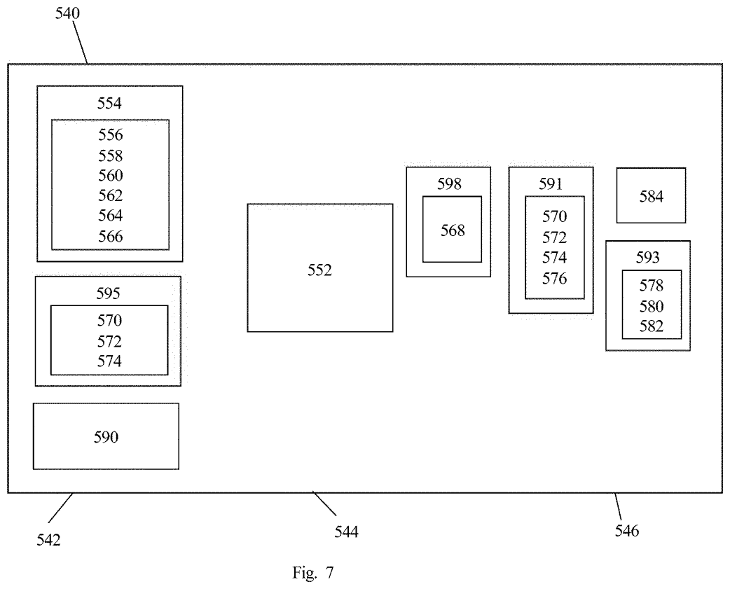

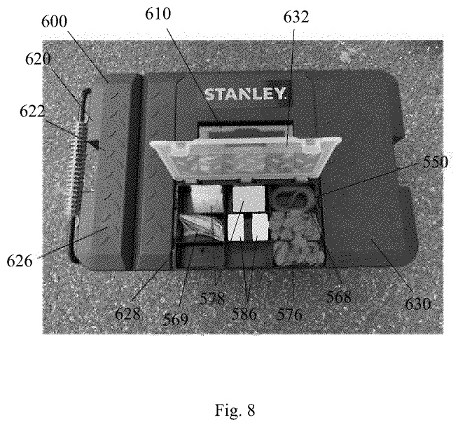

As shown in FIG. 7, container 552 may be placed in central region 544 of bottom portion 540. In an aspect, container 552 may be a sharps container for disposal of needles. A tube rack 554 may be placed in first side region 542, for example, to the left of container 552.

Tube rack 554 may support one or more of serum separator tubes (SSTs) 556, first tubes 558 (e.g., lavender tubes), second tubes 560 (e.g., blue tubes), and third tubes 562 (e.g., red tubes). In another aspect, tube rack 554 may also include writing implements, such as Sharpie™ markers 564, and/or pens 566.

Supplies 550 may be included to maintain specimen integrity. Tube selection may be based on the test for which specimen is being collected. Tubes, for example, first tubes 558, second tubes 560, and third tubes 562, may be color-coded. Tubes may be topped with color coding. Color coding the tubes may be based on the additives contained in the tubes (e.g., anticoagulants, stabilizers, etc.). For example, first tubes 558 may be color-coded with lavender to indicate that first tubes 558 contain ethylenediamine tetraacetic acid (EDTA) additives which prohibit the blood from coagulating and stabilize the specimen to maintain values as they would be in situ. First tube 571 may be used for specimens that require ambient, refrigerated or frozen temperature, but not protection from light. Second tube 573, for example, may be amber transfer tubes that protect the collected specimen from light.

After positioning tube rack 554 in bottom portion 540, one or more of these items may be placed in tube rack 554. In an aspect, approximately one to approximately 15 SSTs 556 may be included, such as approximately 12 SSTs 556; approximately one to approximately 15 first tubes 558 may be included, such as approximately 8 first tubes 558; approximately one to approximately 15 second tubes 560 may be included, such as approximately 9 second tubes 560; approximately one to approximately 15 third tubes 562 may be included, such as approximately three third tubes 562; approximately one to approximately five Sharpie™ markers 564 may be included, such as approximately one Sharpie™ marker 564; and approximately one to approximately five pens 566 may be included, such as approximately one pen 566. On top of tube rack 554 with one or more of these items added, adhesive 586 and/or hand sanitizer 588 may be added. For example, approximately one to approximately five adhesive 586 items may be included, such as approximately two adhesive 586 items; and approximately one four-ounce hand sanitizer 588 may be included.

One or more specimen containers 590 may be placed in first side region 542. For example, approximately one to approximately 15 specimen containers 590 may be included, such as approximately five to approximately 10 specimen containers 590.

For example, approximately five to approximately 15 bands 568 may be included, such as approximately 10 bands 568. A container 595 (e.g., an original needle case with a perforated top) may be placed in first side region 542 in front of tube rack 554 to contain one or more first needles 570 and/or second needles 572. For example, approximately five to approximately 15, such as approximately eight of each of first needles 570 and second needles 572 may be included. In the same container 595 that contains first needles 570 and/or second needles 572, one or more infusion sets 574 may be included. For example, approximately five to approximately 15 infusion sets 574 may be included, such as approximately 10 infusion sets 574.

A container 598 (e.g., a 3″×2″×3″ container) may be placed to the right of container 552 in second side region 546. In an aspect, container 598 may contain one or more bands 568 for specimen collection.

A container 591 (e.g., a 4″×4″ container) may be positioned in second side region 546. In an aspect, container 591 may be positioned to the right of container 598. In an aspect container 591 may contain one or more of first needles 570, second needles 572, infusion sets 574, and gloves 576. Approximately five to approximately 15 gloves 576 may be included in container 591, such as approximately 10 gloves 576. A container 593 (e.g., a 6″×4″×2″ container) may be placed in second side region 546 to contain alcohol swabs 578, gauze 580, and needle holders 582 to the right of container 591 containing first needles 570, second needles 572, infusion sets 574, and gloves 576. Approximately half a pack of gauze 580 may be added. Additionally, approximately five to approximately 30 alcohol swabs 578 may be included, such as approximately 15 alcohol swabs 578; and approximately five to approximately 30 needle holders 582 may be included, such as approximately 10 to approximately 15 needle holders 582. Masks 584 may be placed in second side region 546 behind container 593 containing alcohol swabs 578, gauze 580, and needle holders 582. Approximately one to approximately 15 masks 584 may be included, such as approximately five to approximately 10 masks 584.

Once all of the supplies 550 are added and organized in toolkit 500, a health professional may close bottom portion 540 by adding top portion 520 (FIG. 6). With reference to FIG. 6, one or more chux pads 592 may be disposed on top portion 520 under handle 510. In this way, chux pads 592, which may be the first thing that is used and placed down at a non-medical site, are quickly accessible. For example, approximately one to approximately five chux pads 592 may be included, such as approximately one chux pad 592. Chux pads 592 may be rolled to fit compactly under handle 510. In addition, one or more biohazard containers 594 may be disposed on top portion 520 under handle 510. For example, approximately one to approximately five biohazard containers 594 may be included, such as approximately one biohazard container 594.

SOPs may instruct a health professional to pack items in addition to toolkit 500 for a scheduled specimen collection. As shown in FIGS. 8-11, a health professional may be provided with storage 600. Storage 600 may include a handle 610, a top portion 620, and a bottom portion 640. Top portion 620 may include a top end 622, a bottom end 624, a first side region 626, a central region 628, and a second side region 629. Central region 628 may be recessed in top portion 620. Handle 610 may extend through top portion 620 at central region 628.

Storage 600 may include everything needed to process a collected specimen, such as a specimen collected using toolkit 500. As with toolkit 500 (FIG. 5), storage 600 may be packed prior to prior to a scheduled specimen collection (e.g., the night before). Several supplies 550 may be added to storage 600. These supplies 550 may include both supplies 550, such as supplies 550 specific to the scheduled specimen collection. For example, supplies 550 added to storage 600 may include one or more of tube rack 554, band 568, a bandage 569, a first tube 571 (e.g., an aliquot/transfer tube), a second tube 573 (e.g., an amber transfer tube), a pipette 575, a light protective container 577 (e.g., a bag), alcohol swab 578, a mat 579 (e.g., a floor mat, an extruded vinyl utility runner, etc.), gloves 576, a tachometer 581, a centrifuge 583, a power source 585, adhesive 586, a sanitizing wipe 587, and/or specimen container 590.

Most supplies 550 for a scheduled specimen collection may be contained within storage 600 in bottom portion 640. However, as shown in FIG. 8, one or more supplies 550 may also be received in recessed central region 628 of top portion 620. A top cover 632 may be provided to secure these supplies 550 in top portion 620. One or more bands 568, bandages 569, gloves 576, alcohol swabs 578, and adhesive 586 may be placed in top portion 620. For example, approximately one to approximately 15 bands 568 may be included, such as approximately 10 bands 568; approximately one to approximately 15 bandages 569 may be included, such as approximately five bandages 569; approximately one to approximately 30 gloves 576 may be included, such as approximately 15 gloves 576; approximately one to approximately 30 alcohol swabs 578 may be included, such as approximately 20 alcohol swabs 578; and approximately one to approximately 15 adhesive 586 items may be included, such as approximately two adhesive 586 items.

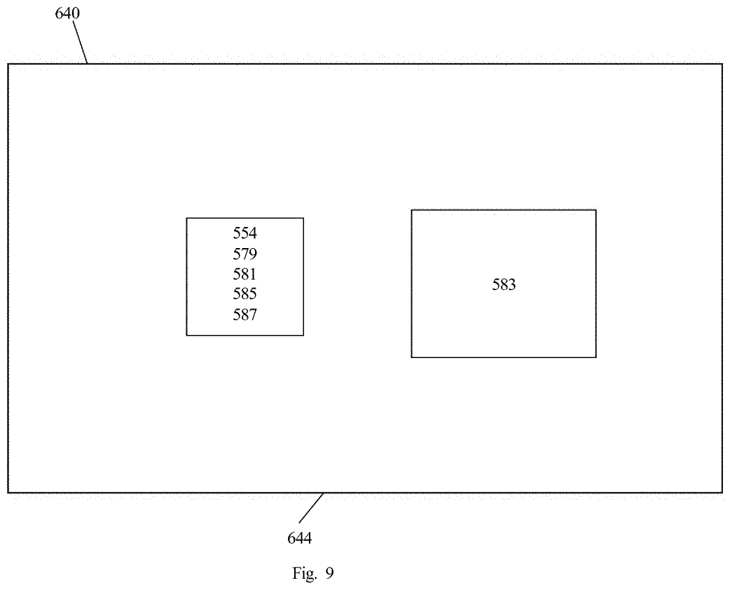

Bottom portion 640 may be covered by top portion 620. FIG. 9 shows bottom portion 640 with top portion 620 (FIG. 8) removed. Bottom portion 640 may include a top region 642 and a bottom region 644. It should be understood that the order of presentation of adding supplies 550 to top portion 620 and bottom portion 640 is not necessarily the order in which supplies 550 may be placed.

Larger and/or heavier supplies 550 may be included in bottom region 644 of bottom portion 640. One or more tube racks 554, mats 579, tachometers 581, centrifuges 583, power sources 585, and sanitizing wipes 587 may be placed in bottom region 644. For example, approximately one to approximately 15 tube racks 554 may be included, such as approximately one tube rack 554; approximately one to approximately two mats 579 may be included, such as approximately one mat 579; approximately one to approximately two tachometers 581 may be included, such as approximately one tachometer 581; approximately one to approximately two centrifuges 583 may be included, such as approximately one centrifuge 583; approximately one to approximately five power sources 585 may be included, such as approximately two power sources 585; and approximately one to approximately 100 sanitizing wipes 587 may be included, such as approximately 75 sanitizing wipes 587.

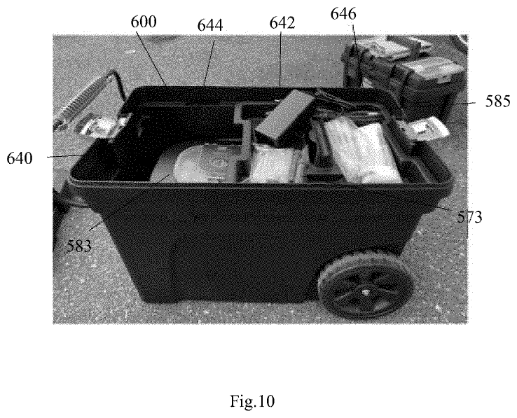

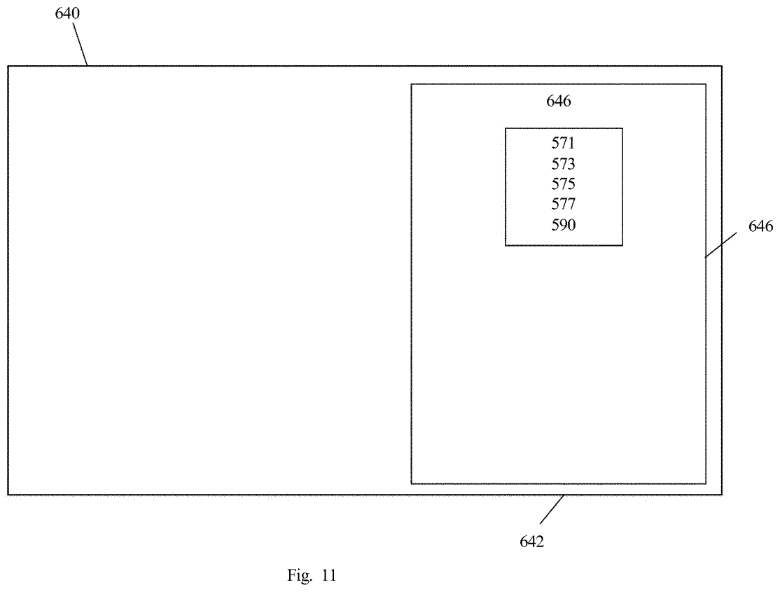

As shown in FIGS. 10-11, a tray 646 may be positioned at least partially across top region 642 of bottom portion 640. In an aspect, additional supplies 550 may be positioned in tray 646. For example, one or more first tubes 571, second tubes 573, pipettes 575, light protective containers 577, and specimen containers 590 may be placed in tray 646. For example, approximately one to approximately 15 first tubes 571 may be included, such as approximately five first tubes 571; approximately one to approximately 15 second tubes 573 may be included, such as approximately two second tubes 573; approximately one to approximately 20 pipettes 575 may be included, such as approximately 15 pipettes 575; approximately one to approximately 10 light protective containers 577 may be included, such as approximately two light protective containers 577; and approximately one to approximately 20 specimen containers 590 may be included, such as approximately 10 specimen containers 590.

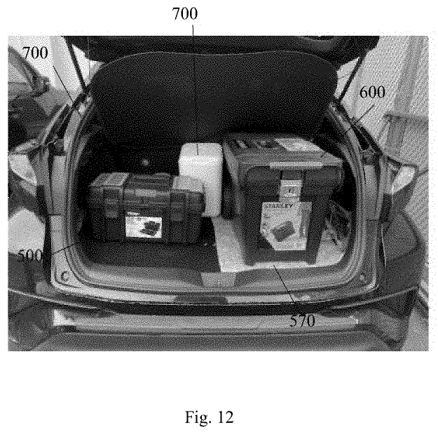

Once both toolkit 500 and storage 600 are ready and supplied as described herein, a health professional may proceed to a scheduled specimen collection appointment. As shown in FIG. 12, a vehicle 10 of the health professional may be loaded with toolkit 500 and storage 600. For example, toolkit 500 and storage 600 may be loaded in a trunk of vehicle 10. In some vehicles 10, toolkit 500 and storage 600 may be maintained in another location, e.g., a backseat. SOPs may instruct the health professional to place toolkit 500 and storage 600 in this way, and also to keep the area free of additional items. Additionally, SOPs may instruct the health professional to balance the weight of vehicle 10 by placing toolkit 500 and storage 600 on opposite sides of vehicle 10. PPE 596 (e.g., gowns, booties, etc.) may also be contained in vehicle 10. Centrifuge 583 may be suitable for use in vehicle 10. For example, centrifuge 583 may be approximately ten pounds or less to fit well in vehicle 10, such as approximately nine pounds. Additionally, vehicle 10 may be required to provide temperature control or to maintain an ambient temperature to facilitate centrifugation and maintain specimen integrity. Accordingly, at least part of vehicle 10 may be considered a centrifuge processing station.

Post-collection of the specimen, mat 579 may be removed from storage 600 and placed in front of the front passenger seat, which may be moved back to provide additional floor space. Centrifuge 583 may be removed from storage 600 and placed flush on mat 579 for use. Mat 579 may provide heat protection and stabilize centrifuge. For example, centrifuge 583 may include a suction cup that secures to mat 579 and reduces vibration. After specimen processing is completed in centrifuge 583, a specimen box 700 containing the specimen may be sealed and placed on mat 579 and centrifuge 583 may be moved back into storage 600. A health professional may be instructed on the speed and temperature of centrifuge 583 to ensure proper operational parameters.

Many tests require centrifugation within a designated time period from the time of collection. Storage 600 facilitates timely centrifugation, allowing for viable specimens processing. Operational perimeters for centrifugation may be defined by the tube manufacturers and by specimen collection requirements for various tests. Ensuring the specimens are centrifuged at the appropriate time intervals, for the appropriate amount of time, and at correct speeds may ensure a properly prepared specimen for testing. Centrifuge 583 selection and control parameter preservation may facilitate the specimen processing while ensuring specimen integrity.

SOPs may instruct health professionals to charge centrifuge power sources prior to a scheduled specimen collection (e.g., the night before). Vehicle 10 and/or portable batteries may be charged to full capacity to facilitate centrifuge operation to collect data at exact timed intervals. Centrifuge 583 cycling may require prolonged power (e.g., from vehicle 10 and/or portable battery power sources) to achieve an appropriate centrifuge speed on each run, where runs may be completed during some or all of a testing day. For example, an appropriate centrifuge speed for a six-minute run may be between approximately 3300 revolutions per minute (RPM) and approximately 4000 RPM, such as between approximately 3310 RPM and approximately 3500 RPM, such as between approximately 3350 RPM and approximately 3450 RPM. Centrifuge 583 selection may be based on speed consistency during each run during a testing day. Other selection criteria to improve mobile phlebotomy performance may include size, weight, and portability. As discussed above, centrifuge 583 may be approximately ten pounds or less to fit well in vehicle 10, such as approximately nine pounds. Additionally, centrifuge 583 may include a suction cup base for adherence to mat 579 for stabilization. Examples of market devices that meet specifications for centrifuge 583 based on testing include the LW Scientific MXU and E8.

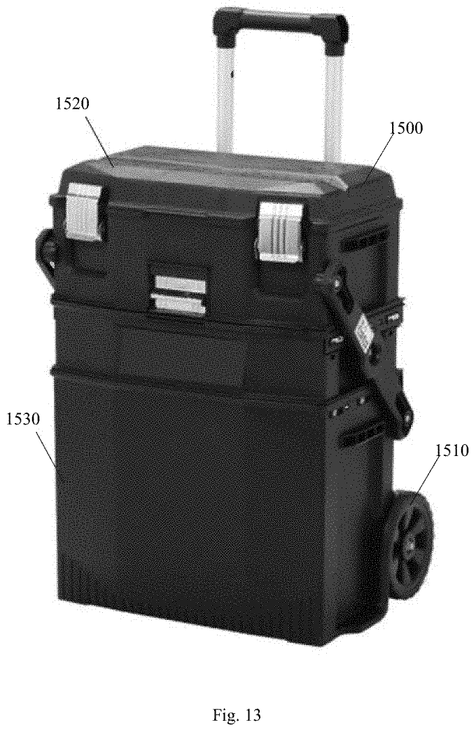

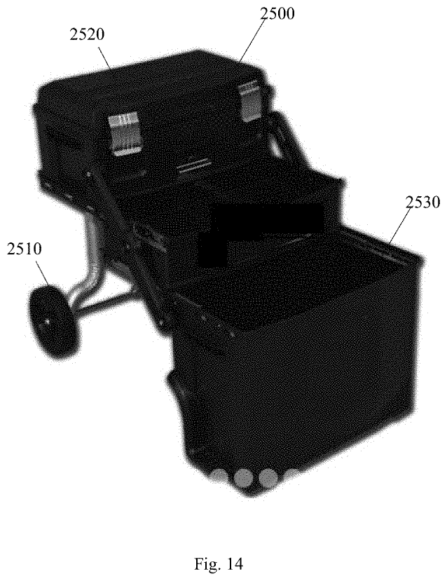

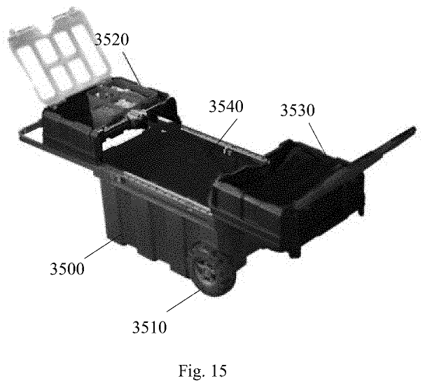

Toolkit 500 (FIG. 5) described herein may be constructed in additional ways. For example, the function and components of toolkit 500 are applicable to a toolkit 1500 (FIG. 13), a toolkit 2500 (FIG. 14), and a toolkit 3500 (FIG. 15) shown and described with reference to FIGS. 13-15.

As shown in FIG. 13, toolkit 1500 may include wheels 1510. Wheels 1510 may allow a heath professional to more easily transport toolkit 1500 between their designated storage location, vehicle 10 (FIG. 12), and/or a non-medical environment. Toolkit 1500 with wheels 1510 may help to transport heavier items as well. Toolkit 1500 may include a top section 1520 and a bottom section 1530, where larger and/or heavier supplies are stored in bottom section 1530. Top section 1520 may be removed to allow access to bottom section 1530 according to some embodiments. Additionally, toolkit 1500 may be organized by utilization with respect to top section 1520 and bottom section 1530. For example, frequently used supplies may be stored in top section 1520, which is more accessible to the health professional. Less used supplies and/or backup supplies may be stored in bottom section 1530. Each of top section 1520 and bottom section 1530 may include dividers and/or other organizational structures. The dividers may be removable for customization.

Similar to toolkit 1500, toolkit 2500 shown in FIG. 14 also includes wheels 2510, a top section 2520, and a bottom section 2530. Top section 2520 may be slideable to allow access to bottom section 2530 according to some embodiments. In this way, a health professional may access bottom section 2530 without having to lift top section 2520. Toolkit 3500 shown in FIG. 15 may also include sliding storage. A first side 3520 and a second side 3530 may be slideable outwardly for access to a bottom portion 3540. Each of first side 3520 and second side 3530 may be lockable to prevent unintended sliding and/or access to bottom portion 3540.

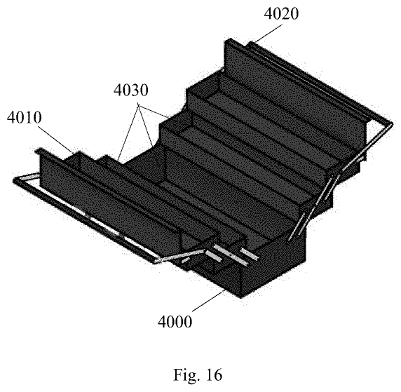

Any of toolkit 500 (FIG. 5), toolkit 1500 (FIG. 13), toolkit 2500 (FIG. 14), and toolkit 3500 may include one or more cantilevered trays 4000 shown in FIG. 16. Cantilevered trays 4000 may facilitate quick and easy access to supplies. Two cantilevered trays 4000 may be provided on a first side 4010 and a second side 4020. Alternatively, one cantilevered tray 4000 on one side may be provided. Each cantilevered tray 4000 may include one or more compartments 4030 for storage. Compartments 4030 may be organized by utilization. For example, frequently used supplies may be stored in higher compartments 4030, which are more accessible to a health professional. Less used supplies and/or backup supplies may be stored in lower compartments 4030. Additionally, compartments 4030 may include dividers and/or other organizational structures. The dividers may be removable for customization.

Referring again to FIG. 1, a specimen collection health professional may access a specimen collection completion module 180 to document and process results from the scheduled specimen collection. Specimen collection completion module 180 may include specimen documentation 181 for documenting the results of the scheduled specimen collection. A health professional may provide digital evidence of the specimen collection to specimen documentation 181. For example, a health professional may provide completion notes or photographs of the collected specimen and attach it to the job or event for the eligible specimen order 152. Specimen documentation 181 may retrieve the results of the specimen collection and monitor for specimen collection completion.

Specimen collection completion module 180 may store the necessary steps for specimen collection completion. These steps may be customized based on scheduled specimen collection for eligible specimen order 152. Specimen documentation 181 may determine whether all the appropriate results of the specimen collection have been retrieved and/or whether all the necessary steps of the specimen collection have been performed, which may provide an audit trail for every step of the specimen collection. Specimen collection completion module 180 may also include specimen collection customization 182 for post specimen collection processing.

Post specimen collection, a health professional may drop off the specimen collected for eligible specimen order 152 at a designated drop-off location or laboratory. In some embodiments, the designated drop-off location may be specified by the type of collection. For example, the drop-off location may be with a mail carrier. In some embodiments, specimen collection results and/or an indication of collection completion may be transmitted to patient system 160 to display to the patient the results of the specimen collection within a patient portal or HIPAA compliant interface. For example, the specimen collection result transmitted to patient system 160 may be an electronic message or notification indicating that collection has been completed. Specimen collection results may be information that a patient accesses in a patient portal post specimen collection.

A health professional may then close eligible specimen order 152 by designating the request with a completion test code 183 to indicate that the specimen collection and drop-off has been completed. Specimen collection completion module 180 may transmit completion test code 183 to eligibility processing module 151, specimen order dispatch module 170, and/or patient system 160. Eligibility processing module 151, specimen order dispatch module 170, and/or patient system 160 may update the test code 130 associated with eligible specimen order 152 to indicate that the specimen collection has been completed.

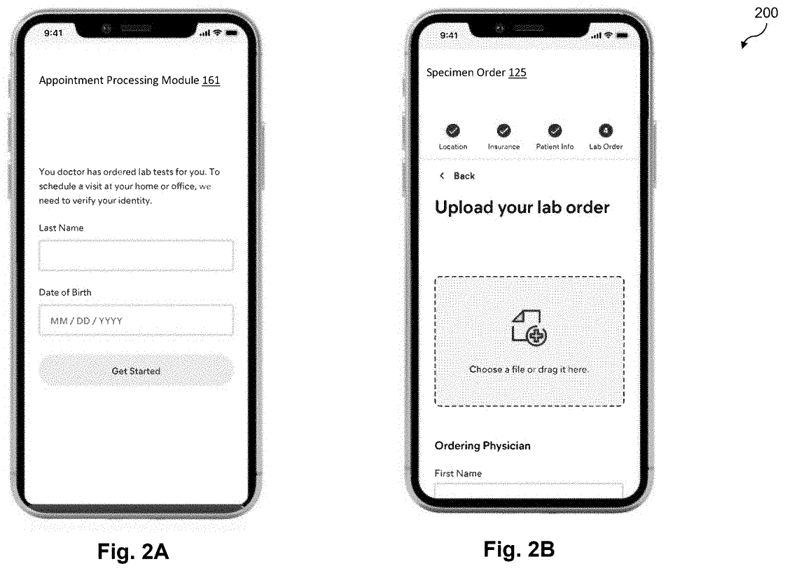

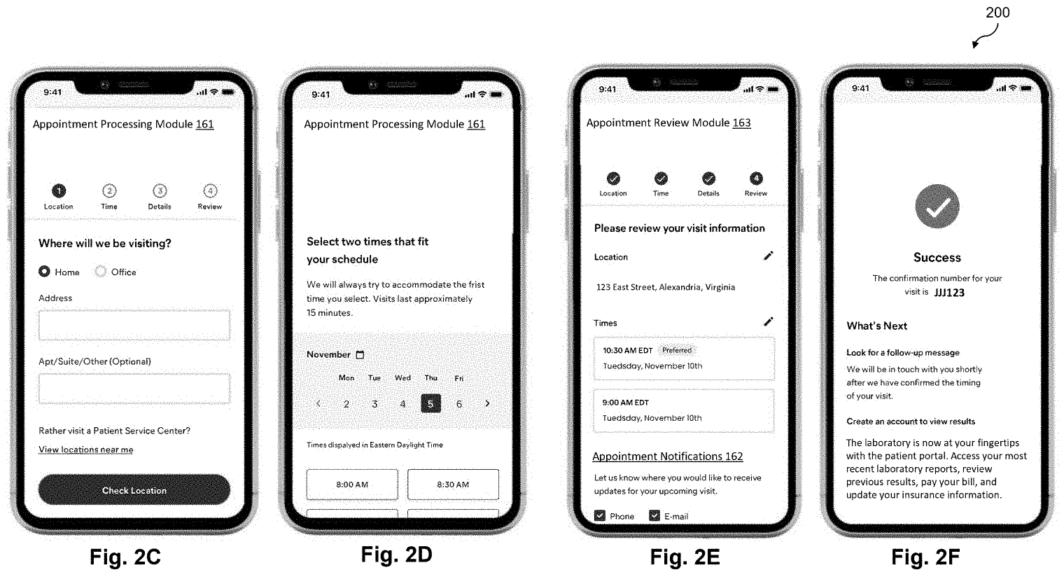

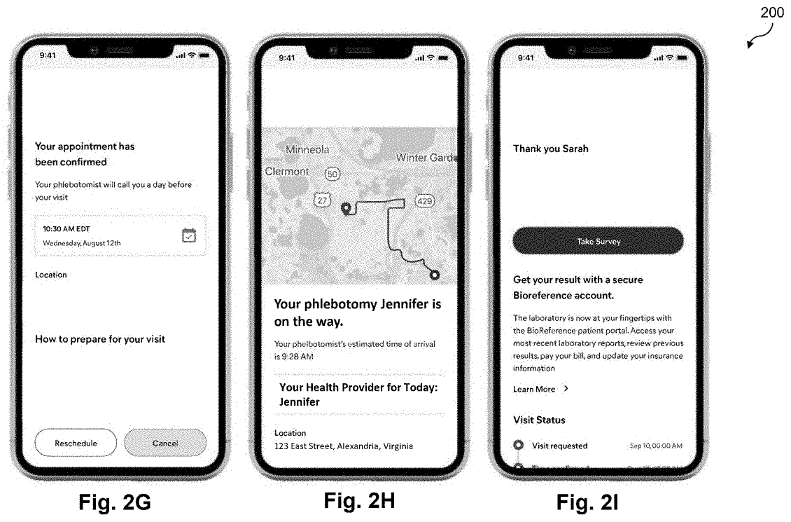

FIGS. 2A-2I illustrate example graphical user interfaces, which may be displayed via patient system 160. As discussed above, processing of a specimen collection order may be initiated by patients and/or with the use of test code 130. In the case of test code 130, laboratory information system 120 may retrieve a specimen order 125 from a healthcare provider 110. Healthcare provider 110 may input an adaptive specimen collection order (e.g., specimen order 125) including a test code 130 into laboratory information system 120. Test code 130 may be a unique test code for system 100 to adaptively route the associated specimen order 125 based on its specific processing needs. Test code 130 may include, but is not limited to, laboratory record data store test code 131, automated specimen processing module test code 132, or specimen collection module test code 133.

Specimen processing module 150 may retrieve a specimen order 125 from a data store containing a collection of specimen orders 125. After specimen processing module 150 retrieves the specimen order 125, eligibility processing module 151 may determine whether the specimen order 125 is an eligible specimen order 152 based on whether its test code 130 is a qualifying test code. Once eligibility processing module 151 determines that specimen order 125 is an eligible specimen order 152, specimen processing module 150 may transmit a communication notification 153 to a patient (e.g., to patient system 160).

Specimen processing module 150 may determine what to include in the communication notification 153 being transmitted to the patent (e.g., to patient system 160) using a communication template 154. For example, specimen processing module 150 may include appointment information 155 in the communication notification 153 based on the communication template 154. The appointment information 155 may include one or more appointment links for the patient to schedule an appointment for the specimen collection related to the eligible specimen order 152. As a security measure, these one or more appointment links may be unique to the associated eligible specimen order 152.

Specimen processing module 150 may also include other information associated with specimen processing module 150 in the communication notification 153 based on the communication template 154. Specimen processing module 150 may also determine how to format the communication notification 153 being transmitted to the patient (e.g., to patient system 160) using the communication template 154. Specimen processing module 150 may also use communication template 154 to tailor the information in the communication notification 153 to a particular patient. Specimen processing module 150 may transmit the communication notification 153 to the patient (e.g., to patient system 160) via email, SMS, or another communication mechanism as would be appreciated by a person of ordinary skill in the art.

As shown in FIG. 2A, the example GUI causes a display in the patient system 160 indicating that the healthcare provider has ordered a lab test, “Your doctor has ordered lab tests for you.” This indicates that specimen order 125 is an eligible specimen order 152 based on the retrieved test code 130. The patient can schedule a specimen collection appointment for the eligible specimen order 152. The patient may provide patient information 135 as an input to appointment processing module 161 of patient system 160 to verify the identity of the patient.

In addition to determining whether a specimen order 125 is an eligible specimen order 152 with test code 130, specimen processing module 150 may also process a specimen collection appointment based on a healthcare provider's lab order being submitted by the patient to patient system 160. As shown in FIG. 2B, the example GUI displays a request to the patient to upload a specimen order 125 prepared by a healthcare provider 110. The patient may choose a file containing the specimen order 125 and provide the information of the healthcare provider 110. For example, the patient may use their smartphone to take an image of specimen order 125 prepared by the healthcare provider 110. The patient may then upload the captured image to patient system 160. Once the patient has uploaded the specimen order 125 to patient system 160, patient system 160 transmits the captured image of specimen order 125 to eligibility processing module 151 and eligibility processing module 151 determines whether specimen order 125 submitted by the patient is an eligible specimen order 152.

Whether specimen processing module 150 determines if a specimen order 125 is an eligible specimen order 152 either based on a test code 130 in the specimen order 125 or a patient's submission of the specimen order 125, specimen processing module 150 transmits a communication notification 153 to the patient (e.g., to patient system 160). The communication notification 153 may be an email and/or SMS notification to the patient with appointment links to schedule the visit. As shown in FIG. 2C and FIG. 2D, the patient may choose a date and time of an appointment and choose a location, such as, for example, the patient's home or workplace for the specimen order 125 within the appointment processing module 161. Based on the patient demographics 136, the appointments in the appointment processing module 161 may be presented to the patient as appointment slots based on health professionals assigned to the patient's geographic demographics and the availability of the health professionals to perform specimen collection for the selected date and time. As shown in FIG. 2D, the patient has chosen that the health professional performing the specimen collection will be visiting the patient's home on Thursday, November 5th during one of the designated time slots.

As shown in FIG. 2E, while scheduling the specimen collection, a patient, through the appointment review module 163, may review the details of the upcoming appointment for the specimen collection, opt-in to receive appointment notifications 162, and provide appointment feedback input 164, such as information related to location access and patient needs, to enable a health professional to understand the patient's needs and prepare for the appointment. As shown in FIG. 2E, the patient has changed the appointment schedule from Thursday, November 5th to Tuesday, November 10th. The patient was also able to choose preferred appointment times for the specimen collection for 10:30 AM, as the preferred choice, and 9:00 AM, as the second choice. The patient may have also opted-in for telephone, e-mail, SMS, and/or push notifications, which will enable a patient to be notified of the appointment status 165 of eligible specimen order 152 via telephone and e-mail.

Once the patient has reviewed the appointment information, a patient may then submit the appointment for the eligible specimen order 152. As shown in FIG. 2F, the example GUI displays a confirmation number of the scheduled specimen collection. However, there is also a message that states, “Look for a follow-up message: We will be in touch with you shortly after we have confirmed the timing of your visit.” Although the patient has provided their preferred appointment specimen collection information, specimen processing module 150 may first transmit the appointment information for the eligible specimen order 152 to specimen order dispatch module 170 to confirm whether the specimen collection is serviceable in that region or during the scheduled date and time.

Once the appointment is retrieved, specimen order dispatch module 170 may create a data object and/or a data structure representing a job or event, according to some embodiments. System 100 may determine which health professional is suitable for fulfilling the specimen collection appointment. Order schedule module 171 may consider the availability and other information of a health professional to allocate the job or event corresponding to the retrieved eligible specimen order 152. Order scheduling module 171 may configure the specimen order queue 172 to eliminate any overlapping specimen collection appointments among health professionals.

In addition to information related to health professionals, specimen order dispatch module 170 may also use information retrieved from or related to a patient to configure specimen order deployment 173. Specimen order deployment 173 may consider the location of the patient to configure specimen order queue 172 using region configuration 176. Region level zip codes may be loaded onto the backend of specimen processing module 150 within specimen order dispatch module 170. Specimen order deployment 173 may use availability templates 174 configured to determine whether specimen processing module 150 is serviceable in a particular region based on whether any health professional is available in the region. Using the address and/or zip code retrieved from the patient, region configuration 176 may determine whether a health professional is aligned to the corresponding region.

Based on this determination, specimen order deployment 173 may provide feedback regarding whether specimen processing module 150 is serviceable in the provided region for the scheduled specimen collection. As shown in FIG. 2G, the example GUI displays that specimen order dispatch module 170 confirmed that the appointment for the eligible specimen order 152 is serviceable in Alexandria, Va. at Tuesday, November 10th at 10:30 AM. After specimen order dispatch module 170 transmits the confirmation of the scheduled specimen collection to patient system 160, patient system 160 may include an appointment status 165 and appointment status map 166 to apprise the patient of information related to the upcoming specimen collection. Appointment status 165 may be configured to notify the patient with confirmation details of the appointment for the eligible specimen order 152 and a link to a page with the appointment status 165. As shown in FIG. 2G, the example GUI displays a link for the patient that states, “How to prepare for your visit.” Once, the patient submits a request to access the link, the accessed page may display instructions for the patient related to the upcoming specimen collection appointment and may also include contact information for a health professional. Additionally, appointment status 165 may display the timeline and progress of fulfilling the specimen collection appointment for the eligible specimen order 152.

As shown in FIG. 2H, the example GUI of appointment status 165 may contain appointment status map 166, which displays a map with a route outlining the distance between the health professional performing the specimen collection and the patient's location. Moreover, prior to the scheduled specimen collection appointment, patient system 160 may update appointment status 165 of eligible specimen order 152 as the health professional proceeds to the patient's location. As shown in FIG. 2H, the GUI displaying appointment status 165 provides an update to the patient stating, “Your phlebotomist Jennifer is on the way. Your phlebotomist's estimated time of arrival is 9:28 AM.” The patient may receive notifications as the health professional is en-route to the patient's location to perform the scheduled specimen collection. Patient system 160 may update and display the updated location of the health professional and the patient may track the health professional in real-time within appointment status map 166.

After completing the scheduled specimen collection, a health professional may access a specimen collection completion module 180 to document and process results from the scheduled specimen collection. Specimen collection completion module 180 may include specimen documentation 181 for documenting the results of the scheduled specimen collection. A health professional may provide digital evidence of the specimen collection to specimen documentation 181. For example, a health professional may provide completion notes or photographs of the collected specimen and attach it to the job or event for the eligible specimen order 125. Specimen documentation 181 may retrieve the results of the specimen collection and monitor for specimen collection completion. In some embodiments, specimen collection results may be transmitted patient system 160 to display to the patient the results of the specimen collection within a patient portal or HIPAA compliant interface. As shown in FIG. 2I, the example GUI displays a message to the patient that the patient can access the results of the eligible specimen order 152 in a patient portal.

Additionally, specimen documentation 181 may determine whether all the appropriate results of the specimen collection have been retrieved and/or whether all the necessary steps of the specimen collection have been performed, which may provide an audit trail for every step of the specimen collection. As shown in FIG. 2I, at the bottom of the example GUI, there is a timeline of the appointment status 165. As shown, for example, specimen processing module 150 displays a “Visit Requested” event completed on September 10:00 AM. Accordingly, the patient has a visual summary illustrating the timeline of events for the completed specimen collection for eligible specimen order 152.

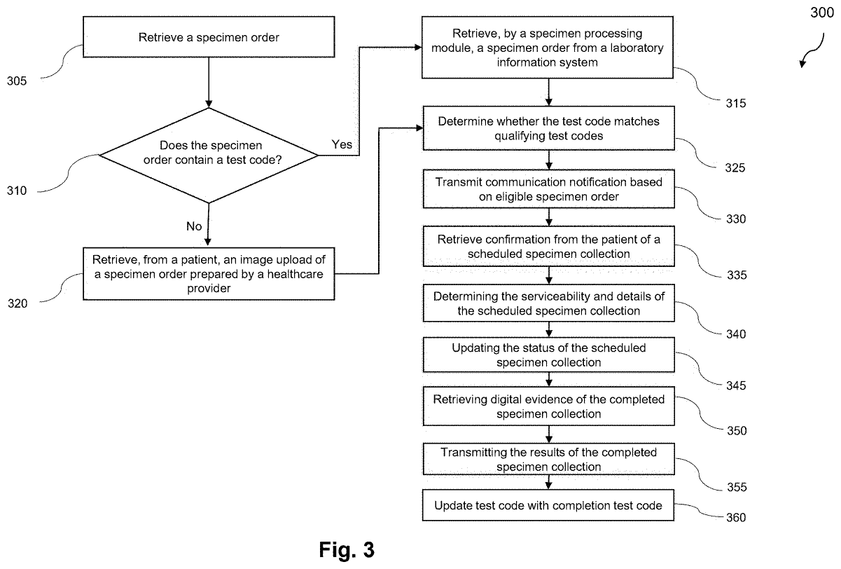

FIG. 3 is a flowchart illustrating a method for adaptive specimen collection order processing, according to some embodiments. FIG. 3 is described with reference to FIGS. 1-2. Method 300 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 3, as will be understood by a person of ordinary skill in the art.

At 305, laboratory information system 120 retrieves a specimen order 125 prepared by a healthcare provider 110. A healthcare provider 110 may access system 100 to place an adaptive specimen collection order (e.g., a specimen order 125) based on a patient's needs. Healthcare provider 110 may place the adaptive specimen collection order by providing relevant information as an input to laboratory information system 120. Laboratory information system 120 may be a graphical user interface through which the healthcare provider 110 submits a specimen order 125 as an input. Specimen order 125 may be a specimen collection order with information including, but not limited to, test code 130, patient information 135, patient demographics 136, and patient contact information 137. Test code 130 may be a unique test code for system 100 to adaptively route a specimen collection order based on the specific processing needs of specimen order 125.

At 310, laboratory information system 120 determines whether specimen order 125 contains a test code 130. Test code 130 may be a unique test code for system 100 to adaptively route a specimen collection order based on the specific processing needs of the specimen collection order (e.g., specimen order 125).

Healthcare provider 110 may input laboratory record data store test code 131, automated specimen processing module test code 132, or specimen collection module test code 133 as part of specimen order 125. Healthcare provider 110 may input other types of test codes as would be appreciated by a person of ordinary skill in the art.

Healthcare provider 110 may input laboratory record data store test code 131 as part of specimen order 125. Laboratory record data store test code 131 may be a test code indicating that specimen order 125 is to be handled by specimen processing module 150. Laboratory record data store test code 131 may be used when a healthcare provider 110 wants to selectively choose when and for which orders specimen orders 125 will be transmitted to specimen processing module 150.

Accordingly, if specimen order 125 contains selective specimen processing test code 131, laboratory information system 120 may transmit that particular specimen order 125 to specimen processing module 150. Therefore, because laboratory record data store test code 131 is provided within specimen order 125, healthcare provider 110 may need to provide a laboratory record data store test code 131 within another specimen order 125 if that other specimen order 125 is to be handled by specimen processing module 150.

Specimen order 125 may also contain an automated specimen processing module test code 132. In many circumstances, healthcare providers 110 may transmit specimen order requests 125 to different systems. However, if a healthcare provider 110 anticipates that all specimen order requests 125 will ultimately be processed by specimen processing module 150, healthcare provider 110 may provide automated specimen processing module test code 132. As a result, healthcare provider 110 may not need to provide a laboratory record data store test code 131. Instead, healthcare provider 110 may provide only automated specimen processing module test code 132. Accordingly, when laboratory information system 120 receives a specimen order 125, laboratory information system 120 may determine that the specimen order 125 is associated with a customer or account number of the healthcare provider 110. Laboratory information system 120 may determine that the healthcare provider 110 provided automated specimen processing module test code 132. As such, laboratory information system 120 may automatically route the specimen order 125 to specimen processing module 150. As a result, the healthcare provider 110 may not need to provide a test code 130 for every future specimen order 125. This configuration can allow for laboratory information system 120 to seamlessly route specimen orders 125 to specimen processing module 150 without requiring any additional information from the healthcare provider 110. By doing so, laboratory information system 120 can require fewer computational resources for routing specimen order 125.

Specimen order 125 may also contain a specimen collection module test code 133. Specimen collection module test code 133 may be used to enable a workflow for specimen collection and drop-off. If healthcare provider 110 anticipates all specimen orders 125 will ultimately be processed through specimen processing module 150, but is limited to specimen collection and drop-off, healthcare provider 110 may provide specimen collection module test code 133 to indicate specimen processing module will only process specimen order 125 for specimen collection and drop-off.

Specimen collection module test code 133 may include a “Date of Collection” parameter as part of specimen order 125 to streamline specimen collection and drop-off. Therefore, if specimen order 125 contains specimen collection module test code 133, laboratory information system 120 may transmit specimen order 125 directly to specimen processing module 150 for the purpose of processing specimen collection and drop-off. The “Date of Collection” parameter may be used to control the dates a patient can actually schedule for the specimen collection.

Specimen processing module 150 may be a master data repository for all laboratory specimen orders 125. Specimen processing module 150 may be a relational database, a NoSQL database or other horizontally scaling database, or any other database adhering to a suitable database design methodology. In some embodiments, specimen processing module 150 may implement a centralized SAN, NAS, redundant array of independent disks, and/or any other configuration of storage devices to supply sufficient storage capacity to store database tables and supporting structures. Sufficient storage may alternatively exist in any other physically attached magnetic storage, cloud storage, or additional storage medium. In some embodiments, specimen processing module 150 may deploy a hard-disk interface, such as ATA, SATA, SCSI, SAS, and/or fibre for interfacing with storage mediums.

If laboratory information system 120 retrieves a specimen order 125 containing a test code 130 for specimen order 125, method 300 proceeds to 315. If laboratory information system 120 retrieves a specimen order 125 that does not contain a test code 130, method 300 proceeds to 320.

At 315, specimen processing module 150 retrieves a specimen order 125 containing a test code 130 from laboratory information system 120. Laboratory information system 120 may have transmitted specimen order 125 to specimen processing module 150.

At 320, specimen processing module 150 retrieves an image upload of specimen order 125 prepared by a healthcare provider 110. The image may be in a non-standardized format for specimen order 125. The image may be uploaded by a patient through patient system 160. The patient may choose a file containing the specimen order 125 and provide the information of the healthcare provider 110. For example, the patient may use their smartphone to take an image of specimen order 125 provided by healthcare provider 110. The patient may then upload the captured image to patient system 160. Once the patient has uploaded the specimen order 125 to patient system 160, patient system 160 may transmit the captured image of specimen order 125 to eligibility processing module 151 and eligibility processing module 151 determines whether specimen order 125 submitted by the patient is an eligible specimen order 152.

In some embodiments, specimen processing module 150 may apply optical character recognition to the image to extract a test code 130 and/or information used to identify a test code 130. For example, this extraction may include formatting the image and/or identifying a portion of the image that identifies the test code 130. This may be the test code 130 itself and/or another representation of the test code. For example, a bar code, QR code, and/or other graphical representation may be used. In some embodiments, specimen processing module 150 may extract more than one test code 130 from the image. In some embodiments, specimen processing module 150 may generate a graphical user interface displaying the image for review and/or validation. For example, an operator, laboratory technician, and/or other health service provider may review the image to identify one or more test codes 130 and/or data used to assign a test code 130. The graphical user interface may also allow the operator to fill an electronic form identifying and/or assigning the one or more test codes 130.

In some embodiments, specimen processing module 150 may first retrieve an image upload of specimen order 125 before method 300 proceeds to 305. The image may be in a non-standardized format for specimen order 125.

At 325, specimen processing module 150 determines whether the retrieved test code 130 associated with the specimen order 125 matches qualifying test codes. By using test code 130, system 100 may adaptively route a specimen collection order based on the specific needs of a healthcare provider 110 without needing to create a separate system compatible with each laboratory information system 120 for specimen collection order processing.

Eligibility processing module 151 of specimen processing module 150 may determine whether specimen order 125 can be processed based on test code 130. Eligibility processing module 151 may compare the test code 130 to test codes eligible for processing through specimen processing module 150, including, but not limited to, laboratory record data store test code 131, or automated specimen processing module test code 132, specimen collection module test code 133. Eligibility processing module 151 may also determine whether the type of test specified in specimen order 125 (e.g., a test for vitamin D, a test for COVID, a test for glucose level, etc.) can actually be serviced. If eligibility processing module 151 determines the test code 130 matches one of the test codes eligible for processing through specimen processing module 150, eligibility processing module 151 may denote the specimen order 125 as an eligible specimen order 152. An eligible specimen order 152 may represent a specimen order 125 that is capable of being processed.

At 330, specimen processing module 150 transmits a communication notification 153 based on the eligible specimen order 152 to a patient (e.g., to patient system 160).

Specimen processing module 150 may determine what to include in the communication notification 153 being transmitted to the patient (e.g., to patient system 160) using a communication template 154. For example, specimen processing module 150 may include appointment information 155 in the communication notification 153 based on the communication template 154. The appointment information 155 may include one or more appointment links for the patient to schedule an appointment for the specimen collection related to the eligible specimen order 152. Specimen processing module 150 may include other information associated with specimen processing module 150 in the communication notification 153 based on the communication template 154. Specimen processing module 150 may use communication template 154 to tailor the information in the communication notification 153 to a particular patient.

Specimen processing module 150 may also determine how to format the communication notification 153 being transmitted to the patient (e.g., to patient system 160) using the communication template 154. Specimen processing module 150 may use communication template 154 to tailor the format of information in the communication notification 153 to a particular patient.

Specimen processing module 150 may transmit the communication notification 153 to the patient (e.g., to patient system 160) via email, SMS, or another communication mechanism as would be appreciated by a person of ordinary skill in the art.

At 335, specimen processing module 150 retrieves confirmation from the patient of a specimen collection appointment for the associated eligible specimen order 152. Patient system 160 may be a graphical user interface through which a patient schedules and manages a specimen order 125. Patient system 160 may include an appointment processing module 161, appointment notifications 162, appointment review module 163, appointment feedback input 164, appointment status 165, and appointment status map 166. In order to schedule a specimen order 125, a patient may access an appointment link via email and/or SMS in the appointment processing module 161. A patient may choose a date and time of an appointment and choose a location, such as, for example, the patient's home or workplace, for the specimen order 125 within the appointment processing module 161. In other words, the patient may provide resource allocation information. Based on the patient demographics 136, the appointments in the appointment processing module 161 may be presented to a patient as appointment slots based on health professionals assigned to the patient's geographic demographics and the availability of the health professionals to perform specimen collection for the selected date and time.

While scheduling the specimen collection, a patient may opt-in to receive appointment notifications 162 that may include notifications related to the eligible specimen order 152. Specimen processing module 150 may configure appointment notifications 162 may retrieve patient's consent to receive appointment notifications for compliance with legal requirements. A patient, through the appointment review module 163, may review the details of the upcoming appointment for the specimen collection, opt-in to receive appointment notifications 162, and provide appointment feedback input 164, such as information related to location access and patient needs, to enable a health professional to understand the patient's needs and prepare for the appointment. A patient may then submit the appointment for the eligible specimen order 152.

At 340, specimen processing module 150 determines the serviceability and details for dispatching the specimen collection appointment corresponding to eligible specimen order 152. Upon the patient submitting the appointment, specimen order dispatch module 170 may retrieve the appointment for the eligible specimen order 152. Specimen order dispatch module 170 may be a backend of system 100 configured to process dispatching the eligible specimen order 152. Specimen order dispatch module 170 may include an order scheduling module 171, specimen order deployment 173, and/or a specimen collection instruction 177. Order schedule module 171 may process eligible specimen orders 152 using a specimen order queue 172. Health professionals may configure incoming eligible specimen orders 152 by configuring the position of these orders in a specimen order queue 172. Health professionals may configure specimen order queues 172 to initiate the operational workflow for the scheduled specimen collection. Health professionals may provide their location, availability, and other information to enhance specimen collection dispatch.

Once the appointment is retrieved, specimen order dispatch module 170 may create an object representing a job or event, according to some embodiments. System 100 may determine which health professional is suitable for fulfilling the specimen collection appointment. Order schedule module 171 may consider the availability and other information of healthcare providers to allocate the job event corresponding to the retrieved eligible specimen order 152. Order scheduling module 171 may configure the specimen order queue 172 to eliminate any overlapping specimen collection appointments among health professionals.

In addition to information related to health professionals, specimen order dispatch module 170 may also use information retrieved from or related to a patient to configure specimen order deployment 173. Specimen order deployment 173 may consider the location of the patient to configure specimen order queue 172 using region configuration 176. Region level zip codes are loaded onto the backend of specimen processing module 150 within specimen order dispatch module 170. Specimen order deployment 173 may use availability templates 174 configured to determine whether specimen processing module 150 is serviceable in a particular region based on whether any health professional is available in the region.

Using the address and/or zip code retrieved from the patient, region configuration 176 may determine whether a health professional is aligned to the corresponding region. Based on this determination, specimen order deployment 173 may provide feedback regarding whether specimen processing module 150 is serviceable in the provided region for the scheduled specimen collection. Region configuration 176 may also customize region configurations to provide flexible scheduling based on the needs of a specific region. Additionally, specimen order deployment 173 may include custom lead time 175 in which regions are set with a custom lead-time that provides additional travel time as a buffer for health professionals traveling to the patient for a scheduled specimen collection.

Additionally, specimen order dispatch module 170 may include a specimen collection instruction 177 to configure the workflow of specimen collection and drop-off. Accordingly, for a health professional to perform the specimen collection, specimen collection instruction 177 may retrieve guidance and instructions for the health professional performing specimen collection, extraction, and or drop-off.

At 345, specimen processing module 150 updates the status of the scheduled specimen collection corresponding to eligible specimen order 152. After processing the appointment for eligible specimen order 152, specimen order dispatch module 170 may transmit information related to the scheduled appointment for eligible specimen order 152 to patient system 160. Patient system 160 may include an appointment status 165 and appointment status map 166 to apprise the patient of information related to the upcoming specimen collection. Appointment status 165 may be configured to notify the patient with confirmation details of the appointment for the eligible specimen order 152 and a link to a page with the appointment status 165. The link may display instructions for the patient related to the upcoming specimen collection appointment and may also include contact information for a health professional. Appointment status 165 may display the timeline and progress of fulfilling the specimen collection appointment for the eligible specimen order 152.

Prior to the scheduled specimen collection appointment, patient system 160 may update appointment status 165 of eligible specimen order 152 as the health professional proceeds to the patient's location. The patient may receive notifications as the health professional is en-route to the patient's location to perform the scheduled specimen collection. The patient may access an appointment status map 166 that displays the location of the health professional in a map. Patient system 160 may update and display the updated location of the health professional and the patient may track the health professional in real-time within appointment status map 166. Patient system 160 may transmit a notification as the health professional arrives at the patient's location. Patient system 160 may also transmit various notifications based on the health professional executing different events for completing the eligible specimen order 152.

At 350, specimen processing module 150 retrieves digital evidence of the completed specimen collection. A health professional may access specimen collection completion module 180 to document and process results from the scheduled specimen collection. Specimen collection completion module 180 may include specimen documentation 181 for documenting the results of the scheduled specimen collection. A health professional may provide digital evidence of the specimen collection to specimen documentation 181. For example, a health professional may provide completion notes or photographs of the collected specimen and attach it to the job for the eligible specimen order 152. The digital evidence may also include typed documentation and/or scanned images of documentation. Specimen documentation 181 may retrieve the results of the specimen collection and monitor for specimen collection completion.

Specimen collection completion module 180 may store the necessary steps for specimen collection completion. These steps may be customized based on scheduled specimen collection for eligible specimen order 152. Specimen documentation 181 may determine whether all the appropriate results of the specimen collection have been retrieved and/or whether all the necessary steps of the specimen collection have been performed, which may provide an audit trail for every step of the specimen collection. Specimen collection completion module 180 may also include specimen collection customization 182 for post specimen collection processing.

Specimen processing module 150 and/or specimen collection completion module 180 may validate received digital evidence to determine the completion or realization of a collection and/or event. For example, specimen collection completion module 180 may apply OCR, fillable forms, and/or validation rules to confirm one or more steps for specimen collection completion or realization. In some embodiments, specimen collection completion module 180 may extract data from images and/or documentation to identify the completion or realization of a particular step. The data may be textual and/or may include a categorization using image processing techniques. For example, specimen collection completion module 180 may extract textual data using OCR. Specimen collection completion module 180 may also apply image classification, such as, for example, classifying an image of a test sample and/or a collection receptacle. In some embodiments, validation may occur based on acknowledgement from a health professional providing an acknowledgment of completion. For example, a health professional may utilize a web application to provide an indication that one or more steps of the specimen collection has been completed. In some embodiments, specimen processing module 150 may generate a graphical user interface displaying the digital evidence for review and/or validation. For example, an operator, laboratory technician, and/or other health service provider may review images and/or document to verify completion or realization of one or more steps of the specimen collection. The graphical user interface may also allow the operator to fill an electronic form indicating the completion or realization of the one or more steps based on operator review.

At 355, specimen processing module 150 transmits the results of the completed specimen collection (e.g., specimen collection results) to laboratory information system 120. Post-collection of the specimen, a health professional may drop off the specimen collected for eligible specimen order 152 at a designated drop-off location or laboratory. In some embodiments, the designated drop-off location may be specified by the type of collection. For example, the drop-off location may be with a mail carrier. In some embodiments, specimen collection results may be transmitted to patient system 160 to display to the patient the results of the specimen collection within a patient portal or HIPAA compliant interface. For example, the specimen collection result transmitted to patient system 160 may be an electronic message or notification indicating that collection has been completed. In some embodiments, the digital evidence may be transmitted to laboratory information system 120.

At 360, specimen processing module 150 updates the test code 130 of the eligible specimen order 152 with a completion test code 183. A health professional may then close the eligible specimen order 152 by designating the request with a completion test code 183 to indicate that the specimen collection and drop-off has been completed. Specimen collection completion module 180 may transmit completion test code 183 to eligibility processing module 151, specimen order dispatch module 170, and/or patient system 160. Eligibility processing module 151, specimen order dispatch module 170, and/or patient system 160 may update the test code 130 associated with the eligible specimen order 152 to indicate that the specimen collection has been completed.

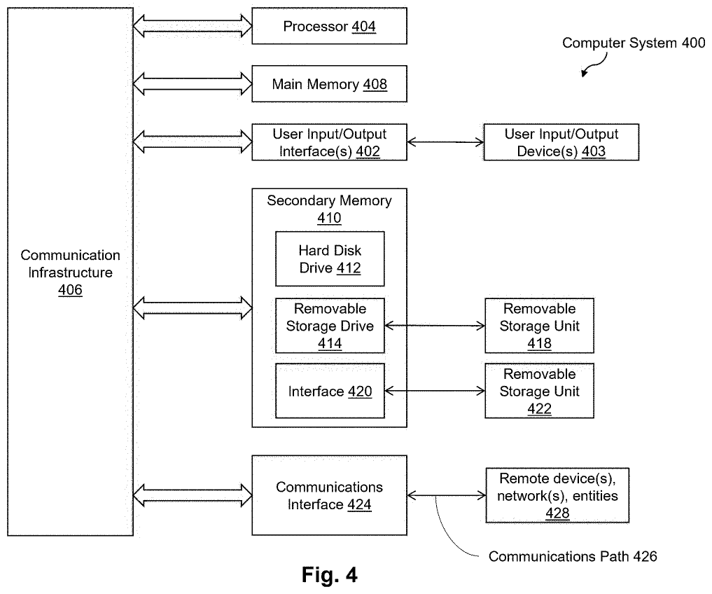

Various embodiments can be implemented, for example, using one or more computer systems, such as computer system 400 shown in FIG. 4. FIG. 4 is described with reference to FIGS. 1-3. Computer system 400 can be used, for example, to implement method 300 of FIG. 3. For example, computer system 400 can implement and execute a set of instructions comprising retrieving a specimen order 125 comprising a test code 130, determining that the test code 130 matches a plurality of test codes eligible for processing the specimen order 125, transmitting and retrieving, to and from a patient system 160, a request to provide information for the processing of an eligible specimen order 152, and generating an event for performing the specimen collection based on the retrieved information in the specimen order dispatch module 170. Computer system 400 can be any computer capable of performing the functions described herein.

Computer system 400 can be any well-known computer capable of performing the functions described herein.

Computer system 400 includes one or more processors (also called central processing units, or CPUs), such as a processor 404. Processor 404 is connected to a communication infrastructure or bus 406.

One or more processors 404 may each be a graphics processing unit (GPU). In some embodiments, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 400 also includes user input/output device(s) 403, such as monitors, keyboards, pointing devices, etc., that communicate with communication infrastructure 406 through user input/output interface(s) 402.

Computer system 400 also includes a main or primary memory 408, such as random access memory (RAM). Main memory 408 may include one or more levels of cache. Main memory 408 has stored therein control logic (i.e., computer software) and/or data.

Computer system 400 may also include one or more secondary storage devices or memory 410. Secondary memory 410 may include, for example, a hard disk drive 412 and/or a removable storage device or drive 414. Removable storage drive 414 may be a floppy disk drive, a magnetic tape drive, a compact disk drive, an optical storage device, tape backup device, and/or any other storage device/drive.

Removable storage drive 414 may interact with a removable storage unit 418. Removable storage unit 418 includes a computer usable or readable storage device having stored thereon computer software (control logic) and/or data. Removable storage unit 418 may be a floppy disk, magnetic tape, compact disk, DVD, optical storage disk, and/any other computer data storage device. Removable storage drive 414 reads from and/or writes to removable storage unit 418 in a well-known manner.

According to an exemplary embodiment, secondary memory 410 may include other means, instrumentalities or other approaches for allowing computer programs and/or other instructions and/or data to be accessed by computer system 400. Such means, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 enables computer system 400 to communicate and interact with any combination of remote devices, remote networks, remote entities, etc. (individually and collectively referenced by reference number 428). For example, communication interface 424 may allow computer system 400 to communicate with remote devices 428 over communications path 426, which may be wired and/or wireless, and which may include any combination of LANs, WANs, the Internet, etc. Control logic and/or data may be transmitted to and from computer system 400 via communication path 426.

In some embodiments, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

1. A computer-implemented method for adaptive specimen collection order processing, comprising:
retrieving, via a specimen processing module, a specimen order for processing a specimen collection occurring at a remote location, wherein the specimen order comprises a test code;
determining, via the specimen processing module, that the specimen order is an eligible specimen order by determining that the test code matches at least one of a plurality of qualifying test codes;
transmitting, over a network via the specimen processing module, to an electronic patient system, a request to provide resource allocation information for processing the specimen collection associated with the eligible specimen order in response to the determining;
generating, via the specimen processing module, an event for performing the specimen collection at the remote location that satisfies the resource allocation information;
retrieving, over the network via the specimen processing module, digital evidence from a health professional that the event was completed;
validating, via the specimen processing module, that the event was completed based on the retrieved digital evidence, wherein the validating comprises verifying completion of one or more steps for the specimen collection by the health professional based on the digital evidence;
transmitting, over the network via the specimen processing module, a specimen collection result to the electronic patient system in response to the validating; and
updating, via the specimen processing module, the eligible specimen order with a completion test code indicating the specimen collection was completed.

2. The method of claim 1, wherein the retrieving the specimen order for processing the specimen collection further comprises:
retrieving, over the network via the specimen processing module, the specimen order for processing the specimen collection from a laboratory information system.

3. The method of claim 1, wherein the test code is automatically routed to the specimen processing module for each specimen order associated with an account number of a healthcare provider, and wherein the retrieving the specimen order for processing the specimen collection further comprises:
retrieving, over the network via the specimen processing module, the specimen order for processing the specimen collection from a laboratory information system.

4. The method of claim 1, wherein the test code is designated for a purpose of collecting and dropping off a specimen, and wherein the retrieving the specimen order for processing the specimen collection further comprises:
retrieving, over the network via the specimen processing module, the specimen order for processing the specimen collection from a laboratory information system.

5. The method of claim 1, further comprising:
transmitting, over the network via the specimen processing module, a status of the generated event to the electronic patient system; and
causing a display, via the specimen processing module, of the status of the generated event in a graphical user interface.

6. The method of claim 5, further comprising:
transmitting, over the network via the specimen processing module, an updated status of the generated event to the electronic patient system; and
causing a display, via the specimen processing module, of the updated status of the generated event in the graphical user interface.

7. The method of claim 6, further comprising:
causing a display, via the specimen processing module, of a location of the health professional performing the specimen collection within a map in the graphical user interface.

8. The method of claim 7, further comprising:
retrieving, over the network via the specimen processing module, an updated location of the health professional performing the specimen collection within the map in the graphical user interface; and
causing a display, via the processing module, of the updated location of the health professional performing the specimen collection within the map in the graphical user interface in real-time.

9. The method of claim 1, further comprising:
transmitting, over the network via the specimen processing module, the digital evidence to a laboratory system.

10. A system for adaptive specimen collection order processing, comprising:
a memory; and
at least one processor coupled to the memory and configured to:
retrieve a specimen order for processing a specimen collection occurring at a remote location, wherein the specimen order comprises a test code;
determine that the specimen order is an eligible specimen order by determining that the test code matches at least one of a plurality of qualifying test codes;
transmit, over a network to an electronic patient system, a request to provide resource allocation information for processing the specimen collection associated with the eligible specimen order in response to the determining;
generate an event for performing the specimen collection at the remote location that satisfies the resource allocation information;
retrieve, over the network from a health professional, digital evidence that the event was completed;
validate that the event was completed based on the retrieved digital evidence, wherein to validate the event the at least one processor is further configured to verify completion of one or more steps for the specimen collection by the health professional based on the digital evidence;
transmit, over the network, a specimen collection result to the electronic patient system in response to the validating; and
update the eligible specimen order with a completion test code indicating the specimen collection was completed.

11. The system of claim 10, wherein to retrieve the specimen order for processing the specimen collection, the at least one processor is further configured to:
retrieve, over the network, the specimen order for processing the specimen collection from a laboratory information system.

12. The system of claim 10, wherein the test code is automatically routed to the specimen processing module for each specimen order associated with an account number of a healthcare provider, and wherein to retrieve the specimen order for processing the specimen collection, the at least one processor is further configured to:
retrieve, over the network, the specimen order for processing the specimen collection from a laboratory information system.

13. The system of claim 10, wherein the test code is designated for a purpose of collecting and dropping off a specimen, and wherein to retrieve the specimen order for processing the specimen collection, the at least one processor is further configured to:
retrieve, over the network, the specimen order for processing the specimen collection from a laboratory information system.

14. The system of claim 10, wherein the at least one processor is further configured to:
transmit, over the network, a status of the generated event to the electronic patient system; and
cause a display of the status of the generated event in a graphical user interface.

15. The system of claim 14, wherein the at least one processor is further configured to:
transmit, over the network, an updated status of the generated event to the electronic patient system; and
cause a display of the updated status of the generated event in the graphical user interface.

16. The system of claim 15, wherein the at least one processor is further configured to:
cause a display of a location of a health professional performing the specimen collection within a map in the graphical user interface.

17. The system of claim 16, wherein the at least one processor is further configured to:
retrieve, over the network, an updated location of the health professional performing the specimen collection within the map in the graphical user interface; and
cause a display of the updated location of the health professional performing the specimen collection within the map in the graphical user interface in real-time.

18. The system of claim 10, wherein the at least one processor is further configured to:
transmit, over the network, the digital evidence to a laboratory system.

19. A non-transitory computer-readable medium having instructions stored thereon that, when executed by at least one computing device, cause the at least one computing device to perform operations for adaptive specimen collection order processing comprising:
retrieving a specimen order for processing a specimen collection occurring at a remote location, wherein the specimen order comprises a test code;
determining that the specimen order is an eligible specimen order by determining that the test code matches at least one of a plurality of qualifying test codes;
transmitting, over a network to an electronic patient system, a request to provide resource allocation information for processing the specimen collection associated with the eligible specimen order in response to the determining;
generating an event for performing the specimen collection at the remote location that satisfies the resource allocation information;
retrieving, over a network digital evidence from a health professional that the event was completed;
validating that the event was completed based on the retrieved digital evidence, wherein the validating comprises verifying completion of one or more steps for the specimen collection by the health professional based on the digital evidence;
transmitting, over the network, a specimen collection result to the electronic patient system in response to the validating; and
updating the eligible specimen order with a completion test code indicating the specimen collection was completed.

20. The non-transitory computer-readable medium of claim 19, wherein the operations further comprise:
transmitting a status of the generated event to the electronic patient system;
causing display of the status of the generated event for the specimen collection in a graphical user interface;
transmitting an updated status of the generated event to the electronic patient system;
causing display of the updated status of the generated event in the graphical user interface;
causing display of a location of a health professional performing the specimen collection within a map in the graphical user interface;
retrieving an updated location of the health professional performing the specimen collection within the map in the graphical user interface; and
causing display of the updated location of the health professional performing the specimen collection within the map in the graphical user interface in real-time.
